(19) United States
(12) Patent Application Publication
(10) Pub. No.: US 2017/0226075 A1
(43) Pub. Date: Aug. 10, 2017

(54) Synthesis of Non-Ionic Surfactants From 5-Hydroxymethyl-2-Furfural, Furan-2,5-Dimethanol and Bis-2,5-Dihydroxymethyl-Tetrahydrofurans

(71) Applicant: Archer Daniels Midland Company, Decatur, IL (US)

(72) Inventors: Kenneth Stensrud, Decatur, IL (US); Lori Wicklund, Argenta, IL (US)

(21) Appl. No.: 15/503,329

(22) PCT Filed: Aug. 19, 2015
(86) PCT No.: PCT/US15/45807

§ 371 Date: Feb. 10, 2017

Related U.S. Application Data
(60) Provisional application No. 62/039,091, filed on Aug. 19, 2014.

(51) Int. Cl. C07D 307/12; C07D 307/42; C07D 307/52; C07D 307/14

(52) U.S. Cl. CPC C07D 307/12; C07D 307/14; C07D 307/42; C07D 307/52; C12Y 202/00; C12R 1/72

(57) ABSTRACT

The present disclosure is directed to methods of making non-ionic, amphiphilic surfactants from 5-hydroxymethyl-2-furfural, furan-2,5-dimethanol and bis-2,5-dihydroxymethyltetrahydrofurans and the novel compounds that are made from those methods.

### CROSS REFERENCE TO RELATED APPLICATION

The present application claims priority to United States Provisional Patent Application No. 62/039,091, filed Aug. 19, 2014.

### FIELD OF INVENTION

The present application relates to the preparation of amphiphilic compounds from bio-derived molecules. In particular, the present disclosure describes preparation of derivatives from sugar-derived alcohols and glycols.

### BACKGROUND OF THE INVENTION

The ineluctable depletion of petroleum reservoirs, which have served as the primary, abundant, and inexpensive source of innumerable specialty and commodity chemicals for over a century, has obliged scientists around the world to implement cutting edge research programs in search of more sustainable surrogates, particularly those derived from biomass. A subset of biomass is a family of ubiquitous, divergent, high-energy materials termed carbohydrates or sugars (i.e., hexoses and pentoses), that can readily be transformed into versatile platforms.

One such precursor, readily made from the acid-catalyzed dehydration of fructose, is the compound 5-hydroxymethyl-2-furfural (FIG. 1).

HMF exemplifies a bifunctional substrate, suitable for the generation of a plethora of furanic analogs that serve as starting materials for further chemical transformations. Additionally, these materials are plausible surrogates for substitutes to the pervasive, benzene-based aromatic compounds that are entirely reliant on oil reserves for continued production. Recent advances in process technology have afforded greater access to HMF at the commercial scale, and such has allowed for innovative endeavors into the synthesis of novel analogs to be executed within a reasonable budget. The thermo-oxidative lability of HMF, however, has limited uses as a chemical per se, other than as a source for making derivatives. Moreover, HMF itself is rather unstable and tends to polymerize and or oxidize with prolonged storage. Due to the instability and limited applications of HMF itself, studies have broadened to include the synthesis and purification of a variety of HMF derivatives.

While the intrinsic reactivity of HMF is challenging and has received limited exposure in the research world to date, there has been some work done Monika Krsytof and others (ChemSusChem 2013, 6, 630-634). Krystof et al. explores a lipase-catalyzed (trans)esterification of HMF with different acyl donors. Most of these experiments were done in solvent free conditions (neat) and once done, separation of the unreacted HMF and HMF esters were performed by using deep-eutectic solvents (DES) as separation agents.

The selective catalytic reduction of HMF aldehyde engenders furan-2,5-dimethanol (FDM Part B of FIG. 2), a highly crystalline material that has received heretofore limited attention as a value added building block, because of its commercial unavailability and substantially high cost. The exploitation of this glycol as a pre-polymer unit, a robust precursor to furan-2,5-dicarboxylic acid (FDCA), posited to be a feasible surrogate to terephthalic acid, or as underscored in the present invention, a scaffold to manifold amphipathic materials, is garnering a significant amount of attention in the global bio-based realm of research.

Catalyzed, exhaustive hydrogenation of HMF, which is shown in FIG. 3, under mild conditions generates THF-diols, also known by their IUPAC names: ((2R,5S)-tetrahydrofuran-2,5-diyl)dimethanol B of FIG. 3 and ((2S,5S)-tetrahydrofuran-2,5-diyl)dimethanol C of FIG. 3 (collectively regarded as 2,5-bishydroxymethyl-tetrahydrofurans (also referred to herein as bHMTHFs)), in a 90:10 cis:trans diastereomeric mixture.

bHMTHFs are versatile molecules that when modified can serve as a substitute for a variety of structurally analogous molecules that have conventionally been derived from petroleum-based sources. Hitherto, research for chemical derivatives using bHMTHFs has received limited attention due in part to the great cost and commercial unavailability of the compounds. Recently, a need has arisen for a way to unlock the potential of bHMTHFs and their derivative compounds, as these chemical entities have gained attention as valuable glycolic antecedents for the preparation of polymers, solvents, additives, lubricants, and plasticizers, etc. Furthermore, the inherent, immutable chirality of bHMTHFs makes these compounds useful as potential species for pharmaceutical applications or candidates in the emerging chiral auxiliary field of asymmetric organic synthesis. Given the potential uses, a cost efficient and simple process that can synthesize derivatives from bHMTHFs would be appreciated by manufacturers of both industrial and specialty chemicals alike as a way to better utilize biomass-derived carbon resources.

### SUMMARY OF INVENTION

The present disclosure describes the use of dehydrated monosaccharides and reduction products thereof in the preparation of non-ionic amphiphiles that can be employed as green surfactants. In particular embodiments, the present invention pertains to compounds such as a furan or tetrahydrofuran compound selected from the group consisting of:

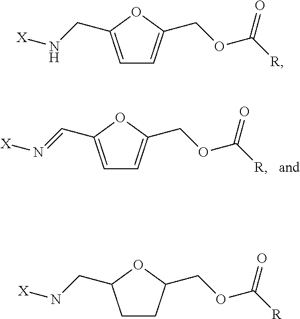

wherein R is a carbon side chain of a fatty acid and X is an organic substituent having sufficient hydrogen bonding capacity to make the compound amphiphilic. In exemplary embodiments, the carbon side chain of one of these compounds is between 8 and 30 carbons.

Other embodiments include compounds such as a furan or tetrahydrofuran compound selected from the group consisting of:

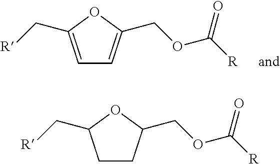

wherein R is a carbon side chain of a fatty acid and R′ is a sulfonate ester moiety. In exemplary embodiments, the carbon side chain of one of these compounds is between 8 and 30 carbons. In a certain embodiment, the sulfonate ester moiety is created by a sulfonating agent selected from the group consisting of p-toluenesulfonyl (tosyl), methanesulfonyl, (mesyl), ethanesulfonate (esyl), benzenesulfonate (besyl), p-bromobenzenesulfonate (brosyl), and triflouromethanesulfonic anhydride (triflate).

In another aspect, there is provided a method of making a furan imine compound of the formula

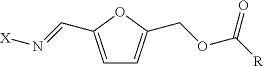

comprising contacting a HMF fatty acid ester with a primary amine to form the imine of the HMF fatty acid ester. In certain embodiments, a HMF fatty acid ester is made by contacting HMF with a fatty acid in the presence of a lipase enzyme. In an exemplary embodiment, the lipase enzyme is Candida Antarctica B lipase. In a certain embodiment, the contacting of the HMF fatty acid ester is carried out at a temperature of about 30° C. to about 100° C., more specifically 40° C. to about 75° C., and most specifically of about 50° C.

In certain embodiments, the contacting of the HMF fatty acid ester with the primary amine to form the imine compound is done in the presence of a polar solvent selected from the group consisting of acetonitrile, dimethylformamide, dimethylacetamide, dimethylsulfoxide, isopropanol, methanol, and ethanol. A further embodiment is where the imine compound is reduced with a reducing agent to form a corresponding amine of the HMF fatty acid ester. In certain embodiments, the reducing agent is a hydride, more specifically a borohydride selected from the group consisting of sodium cyanoborohydride, lithium borohydride, calcium borohydride, magnesium borohydride, and sodium borohydride. In exemplary embodiments, the reduction of the imine fatty acid ester is done at a temperature from about −20° C. to about 26° C., more specifically from about −10° C. to about 10° C., most specifically of about 0° C. In certain embodiments the reduction of the imine fatty acid ester is done in the presence of a polar solvent selected from the group consisting of dimethylsulfoxide, dimethylformamide, dimethylacetamide, methanol, ethanol, isopropanol, tetrahydrofuran, and acetone.

In another aspect, there is provided a method of making a compound of the formula selected from the group consisting of

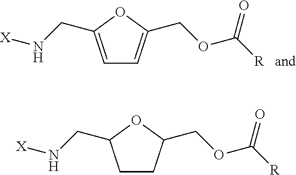

comprising contacting dihydroxymethylfuran or a tetrohydrofuran fatty acid ester with a sulfonating agent to form a sulfonate ester moiety making a sulfonated fatty acid ester, and contacting the sulfonated ester moiety with a primary amine to displace said sulfonated ester moiety with the primary amine in the presence of a nucleophilic base selected from the group consisting of dimethylaminopyridine, imidazole, pyrazole, and pyridine. In certain embodiments, the dihydroxymethylfuran or tetrohydrofuran fatty acid ester is made by contacting the dihydroxymethylfuran or tetrahydrofuran with a fatty acid in the presence of a lipase enzyme. In exemplary embodiments the lipase enzyme is Candida Antarctica B lipase.

In certain embodiments the contacting of the dihydroxymethylfuran or tetrohydrofuran fatty acid ester with the sulfonating agent is done in the presence of an organic solvent selected from the group consisting of chloroform, tetrahydrofuran, acetone, benzene, diethyl ether, and methylene chloride. In exemplary embodiments the fatty acid ester is contacted with the sulfonating agent to form a sulfonate ester at a temperature of from about −20° C. to about 26° C., more specifically, from about −10° C. to about 10° C., most specifically of about 0° C.

In certain embodiments the contacting of the sulfonated fatty acid ester with the primary amine is done in the presence of a polar solvent selected from the group consisting of dimethylsulfoxide, dimethylformamide, dimethylacetamide, N-methylpyrrolidone, acetonitrile, methanol, ethanol, and acetone. An exemplary embodiments the sulfonated fatty acid ester is contacted with said primary amine at a temperature from about 30° C. to about 100° C., more specifically from about 40° C. to about 80° C., most specifically of about 50° C.

Another aspect of the disclosure is a class of esterified furan or tetrahydrofuran compounds selected from the group consisting of

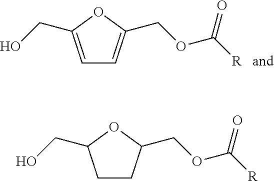

wherein R is a carbon side chain of a fatty acid.

In certain embodiments the carbon side chain of one of these compounds is between 8 and 30 carbons.

In another aspect, there is provided a method of making one of these compounds that consists of contacting a 2,5-dihydroxymethylfuran (FDM) or tetrahydrofuran compound with a fatty acid in the presence of a lipase enzyme under vacuum pressure, more specifically where the vacuum pressure is from about 0.1 torr to about 100 torr, more specifically 2 torr to about 10 torr and most specifically the vacuum pressure is about 5 torr. In exemplary embodiments the furan or tetrahydrofuran compound is contacted with a fatty acid at a temperature is from about 40° C. to about 100° C., more specifically from about 60° C. to about 80° C., most specifically about 70° C. In certain exemplary embodiments the lipase enzyme is Candida Antarctica B lipase.

Additional features and advantages of the present synthesis process and material compounds will be disclosed in the following detailed description. It is understood that both the foregoing summary and the following detailed description and examples are merely representative of the invention, and are intended to provide an overview for understanding the invention as claimed.

### BRIEF DESCRIPTION OF THE DRAWINGS

FIG. 1 depicts HMF synthesis from fructose.

FIG. 2 depicts partial hydrogenation of HMF to furan-2,5-dimethanol (FDM).

FIG. 3 depicts bHMTHFs from the exhaustive reduction of HMF.

FIG. 4 depicts enzymatic acylation of HMF, FDM, bHMTHFs with C₃-C₂₆ carboxylic acids.

FIG. 5 depicts a process for iminatation of an HMF ester.

FIG. 6 depicts an amphipathic HMF ester from imine reduction to amine

FIG. 7 depicts triflation of FDM and bHMTHF mono-esters

FIG. 8 depicts FDM, bHMTHF triflate ester nucleophilic displacement with hydrophilic amines.

FIG. 9 depicts synthesis of (5-(((2-((2-hydroxyethyl)amino)ethyl)amino) methyl)furan-2-yl)methyl palmitate, B as seen in Example 14.

FIG. 10 depicts synthesis of (5-(((2-((2-hydroxyethyl)amino)ethyl)amino) methyl)furan-2-yl)methyl laurate, B as seen in Example 15.

FIG. 11 depicts synthesis of (5-(((2-((2-hydroxyethyl)amino)ethyl)amino) methyl)furan-2-yl)methyl myristate, B as seen in Example 16.

FIG. 12 depicts synthesis of (5-(((2-((2-hydroxyethyl)amino)ethyl)amino) methyl)furan-2-yl)methyl caprate, B as seen in Example 17.

FIG. 13 depicts synthesis of (5-(((2-((2-hydroxyethyl)amino)ethyl)amino) methyl)furan-2-yl)-methyl oleate, B as seen in Example 18.

FIG. 14 depicts synthesis of (5-(((2-(2-hydroxyethoxy)ethyl)amino) methyl)furan-2-yl)-methyl oleate, B as seen in Example 19.

FIG. 15 depicts synthesis of (5-(((2-(2-hydroxyethoxy)ethyl)amino) methyl)furan-2-yl)-methyl palmitate, B as seen in Example 20.

FIG. 16 depicts synthesis of 5-(((2-(2-hydroxyethoxy)ethyl)amino) methyl)furan-2-yl)methyl myristate, B as seen in Example 21.

FIG. 17 depicts synthesis of (5-(((2-(2-hydroxyethoxy)ethyl)amino) methyl)furan-2-yl)-methyl laurate, B as seen in Example 22.

FIG. 18 depicts synthesis of (5-(((2-(2-hydroxyethoxy)ethyl)amino) methyl)furan-2-yl)-methyl caprate, B as seen in Example 23.

FIG. 19 depicts synthesis of (Z)-((2S,5R)-5-((((trifluoromethyl)sulfonyl)oxy) methyl)-tetrahydrofuran-2-yl)methyl nonadec-9-enoate, B as seen in Example 24.

FIG. 20 depicts synthesis of ((2S,5R)-5-((((trifluoromethyl)sulfonyl)oxy) methyl)tetra-hydrofuran-2-yl)methyl tetradecanoate, B as seen in Example 25.

FIG. 21 depicts synthesis of ((2S,5R)-5-(((2-(2-hydroxyethoxy)ethyl)amino) methyl)tetra-hydrofuran-2-yl)methyl oleate, B as seen in Example 26.

FIG. 22 depicts synthesis of ((2S,5R)-5-(((2-(2-hydroxyethoxy)ethyl)amino) methyl)tetra-hydrofuran-2-yl)methyl tetradecanoate, B as seen in Example 27.

FIG. 23 depicts synthesis of ((2S,5R)-5-(((2-((2-hydroxyethyl)amino)ethyl) amino)methyl)-tetrahydrofuran-2-yl)methyl tetradecanoate, B as seen in Example 28.

FIG. 24 depicts ((2S,5R)-5-(((2-((2-hydroxyethyl)amino)ethyl) amino)methyl)tetra-hydrofuran-2-yl)methyl oleate, B as seen in Example 29.

FIG. 25 depicts synthesis of (Z)-(5-((((trifluoromethyl)sulfonyl)oxy) methyl)furan-2-yl)methyl nonadec-9-enoate, B as seen in Example 30.

FIG. 26 depicts synthesis of (5-((((trifluoromethyl)sulfonyl)oxy) methyl)furan-2-yl)methyl dodecanoate, B as seen in Example 31.

FIG. 27 depicts synthesis of (5-(((2-(2-hydroxyethoxy)ethyl)amino) methyl)furan-2-yl)methyl oleate, B as seen in Example 32.

FIG. 28 depicts synthesis of (5-(((2-(2-hydroxyethoxy)ethyl)amino)methyl) furan-2-yl)methyl dodecanoate, B as seen in Example 33.

FIG. 29 depicts synthesis of (5-(((2-((2-hydroxyethyl)amino)ethyl) amino)methyl)furan-2-yl)methyl oleate, B as seen in Example 34.

FIG. 30 depicts synthesis of (5-(((2-((2-hydroxyethyl)amino)ethyl) amino)methyl)furan-2-yl)methyl dodecanoate, B as seen in Example 35.

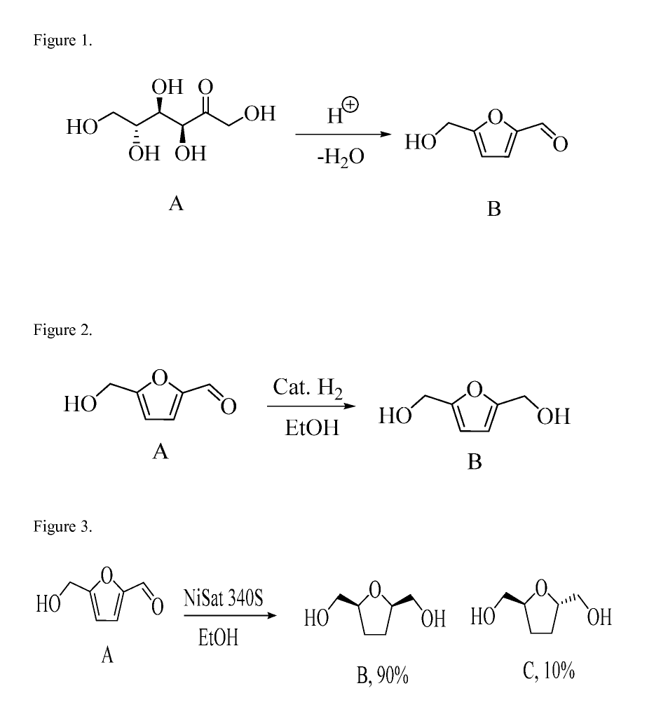

### DEFINITIONS

In order to provide clear and consistent understanding of the specification and claims, including the scope to be given such terms, the following definitions are provided. It is also to be noted that the term “a” and “an” entity, refers to one or more or that entity; for example “a mild reducing agent,” is understood to represent one or more mild reducing agents.

About. In the present application, including the claims, other than in the operating examples or where otherwise indicated, all numbers expressing quantities or characteristics are to be understood as being modified in all instances by the term “about”. Accordingly, unless indicated to the contrary, any numerical parameters set forth in the following description may vary depending on the desired properties one seeks to obtain in the compositions and methods according to the present disclosure. At the very least, and not as an attempt to limit the application of the doctrine of equivalents to the scope of the claims, each numerical parameter described in the present description should at least be construed in light of the number of reported significant digits and by applying ordinary rounding techniques.

Ambient temperature. As used herein, the term ambient temperature refers to the temperature of the surroundings and will be the same as room temperature indoors.

Amphiphile. As used herein, the term amphiphile refers to a chemical compound possessing both hydrophilic (water-loving, polar) and lipophilic (fat-loving) properties. Such a compound is called amphiphilic or amphipathic.

Hydrophilic. As used herein, the term hydrophilic refers to a compound having a tendency to mix with, dissolve in, or be wetted by water.

Overnight. As used herein, the term overnight refers to a time frame of between 10 and 20 hours, typically about 16 hours.

Polarity. As used herein, the term polarity refers to a separation of electric charge leading to a molecule or its chemical groups having an electric dipole or multipole moment. Polar molecules interact through dipole-dipole intermolecular forces and hydrogen bonds.

Neat. As used herein, the term neat refers to the absence of a solvent in a reaction.

Room Temperature. As used herein, the term room temperature refers to a temperature that is between 20° C. and 26° C., with an average of about 23° C.

HMF. As used herein, the abbreviation HMF refers to 5-hydroxymethyl-2-furfural.

FDM. As used herein, the abbreviation FDM refers to furan-2,5-dimethanol.

THF or bHMTHFs As used herein, the abbreviations THF or bHMTHFs are interchangeable and refer to one of the four stereoisomers of bis-2,5-dihydroxymethyltetrahydrofurans.

### DETAILED DESCRIPTION OF INVENTION

Derived primarily from fructose, the sugar alcohol 5-hydroxymethyl-2-furfural (HMF) and its reduced glycol products, 2,5-dihydroxymethylfuran (FDM) and diastereomers 2,5-bishydroxy-methyltetrahydrofuran (bHMTHFs) embody versatile yet relatively unexplored platforms, owing to their commercial unavailability and prodigious costs. As reagents, these molecular species are particularly alluring in that they share a core two-fold functionality, which permits target orientated synthetic approaches to be fostered in the generation of manifold materials with unique chemical properties, such as polymer subunits, plasticizers, lubricants, dispersants, emulsifiers, adhesives coatings, resins, humectants and surfactants.

The present disclosure relates, in part, to the highly efficient, facile two-step preparation of HMF, FDM, and bHMTHF-based amphiphiles, which are novel compounds. The first step to creating these novel compounds is to first create the necessary precursors, which are esterified furan or tetrahydrofuran compounds with a carbon side chain of a fatty acid (R). This carbon side chain can be of 8-26 carbons. On the other side of the ring structure, whether it be of HMF, FDM, or bHMTHF, is a nitrogen atom attached to “X”. For the purposes of this disclosure, “X” is being defined as an organic substituent having sufficient hydrogen bonding capacity to make the corresponding compound that it is attached to amphiphilic. “X” can designate pendant groups containing a sufficient number of electronegative atoms that effectively polarize the group, thus enabling dipole-dipole interactions (hydrogen bonding) and/or ion dipole interactions to occur with an aqueous matrix (hydrophilicity). Conversely, the R group contains a shortage of electronegative atoms, thus is non-polar in nature, and termed “hydrophobic”. Typical, but not limiting examples of such electronegative atoms are oxygen, nitrogen, phosphorus and sulfur and may be found in alcohols, acids, phosphates, sulfoxides and the like.

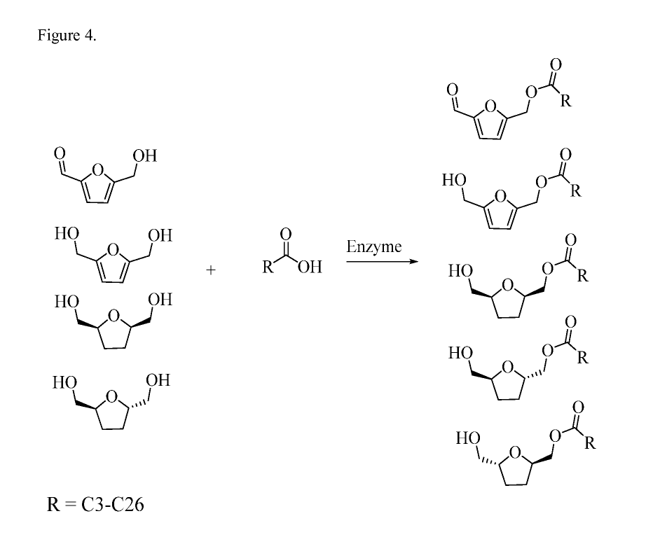

According to an embodiment, the process involves enzymatic acylation of the —OH moiety of HMF, and one —OH moiety of FDM, or THF (bHMTHF), carried out neat in the desired carboxylic acid containing 3 to 26 carbons at a certain temperature under vacuum, as portrayed in FIG. 4 with the enzyme lipase, FDM and oleic acid.

It can be noted that our process stands out and is distinctly different from Krsytof et al. due to the use of short path distillation, also known as molecular distillation, in order to separate the residual unreacted HMF from the HMF esters after the esterification process. Using this type of distillation, the mono- and di-esters are further separated and purified fractions are obtained. Krsytof et al. discloses that this type of distillation would not be recommended or work because of the high temperatures necessary, however, the examples included in this disclosure show that this distillation does work with unexpectedly high efficiency. Additionally, the starting material for the reactions as disclosed by Krystof et al. is HMF. The present disclosure differs from Krsytof et al. in that the starting material is not HMF, but rather partially reduced (FDM) and completely reduced (bHMTHF) compounds.

For the purposes of this invention, 2,5-dihydroxymethylfuran (FDM), although extremely expensive and sometimes hard to find, can be purchased from various supply companies or can be manufactured according to methods that are in the process of being further developed.

Though alcohol acylation can be effectuated by several enzyme classes and variants of the incumbent lipase, Candida Antarctica B lipase (CAB) was deployed in this disclosure as exemplary of the class of lipases for proof of concept. Examples of other lipase enzymes that could be used in this reaction include, but is not limited to Novozyme 435 (an immobilized Candida antarctica lipase B with a permanently open active site), Lipozyme RM IM (a Muchor miehei lipase immobilized with an active site covered by a moveable lid), Lipozyme TM IM (a Thermomyces lanuginosis lipase on porous silica with an active site covered by moveable lid), Lipex 100L (a Thermomyces lanuginosis mutant lipase with enhanced lipid surface absorption with an a active site covered by moveable lid and detergent stable), Palatase 20000 L (a Mucor miehei lipase with an active site covered by a moveable lid), Novozym CALB L (a Candida Articans lipase B with a permanently open active site), Lipozyme TL100 L (a Thermomyces lanuginosis lipase with an active site covered by moveable lid).

CAB acylation can result in copacetic yields of corresponding HMF esters and FDM, and bHMTHF mono esters with carbon side chains of 8-26 carbons, as demonstrated in examples herein.

The acylation process is able to produce HMF esters and FDM, bHMTHF monoesters in reasonably high molar yields of at least 95% from HMF, and at least 40% from FDM and bHMTHF starting materials, typically about 50% or 55% or 60-65% or 70%.

The acylation reaction is usually conducted in the temperature range of 40-100° C., typically 50° C. or 90° C., preferably 60° C. or 80° C., more preferably at about 70° C. Through use of low pressure, the reaction is essentially irreversible, as the water byproduct from condensation is immediately evaporated out of solution. The pressure of the reaction can be from about 0.1 torr to about 100 torr, more specifically from about 2 torr to about 10 torr, most specifically of about 5 torr.

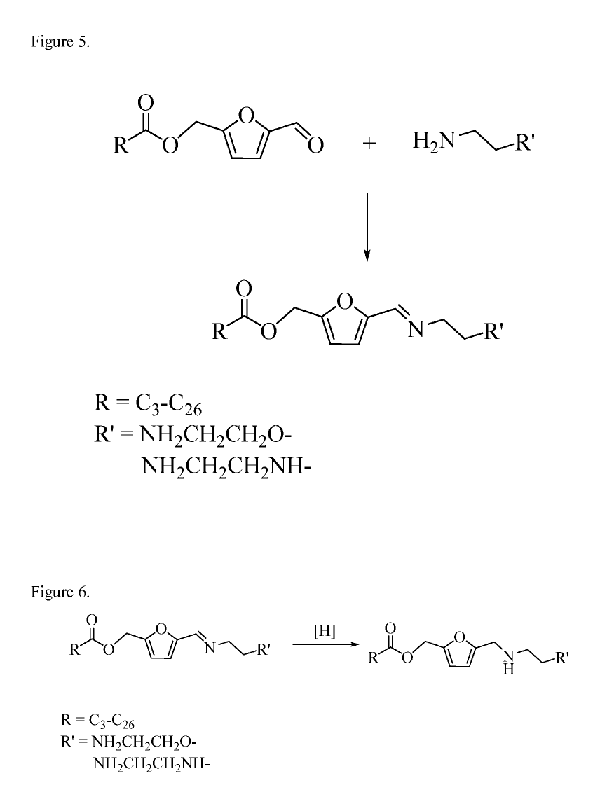

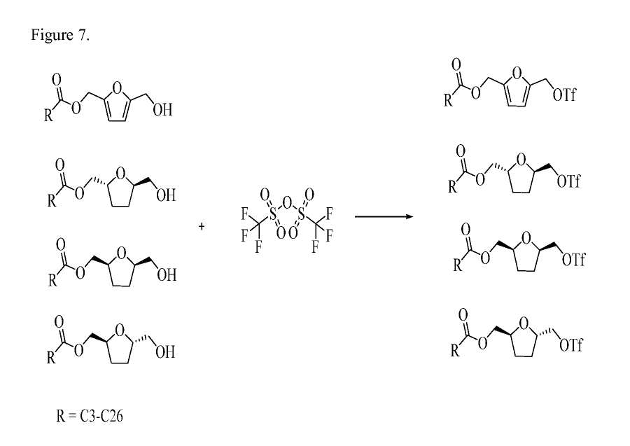

According to another embodiment, the HMF ester that has been made by contacting HMF with a fatty acid in the presence of a lipase enzyme, more specifically the Candida Antarctica B lipase (CAB), undergoes an imination with a hydrophilic amine to afford an imine, as shown in FIG. 5.

According to one embodiment, the imination entails reacting the free aldehyde of HMF with an amine moiety of a hydrophilic species.

The hydrophilic amine consists of a terminal amino moiety (NH₂CH₂CH₂O—, NH₂CH₂CH₂NH—) with sufficient internal oxygen, nitrogen atoms to effectuate water solubility.

The reaction is carried out in an inert, polar solvent, which can be acetonitrile, dimethylformamide, dimethylacetamide, dimethylsulfoxide, isopropanol, methanol, but preferably ethanol as this solvent is a suitable matrix for the supervening imine to amine reduction step.

The reaction occurs at temperatures between 30° C. and 100° C., typically between 40° C. and 75° C., preferably at about 50° C.

Though not isolated in the examples herein, the imine molar yields are quantitative, as determined by ¹H NMR. The imines could be isolated if desired by flash column chromatography.

According to another embodiment, the in situ derived imino precursor is reduced to an amine through application of a mild reducing agent at low temperatures in a suitable, inert solvent, as depicted in FIG. 6 to form the novel amphiphilic compound with an HMF backbone.

The mild reducing agent is drawn from the commonly used borohydride family that includes, but is not limited to, sodium borohydride, sodium cyanoborohydride, lithium borohydride, calcium borohydride, magnesium borohydride.

The reduction is carried out at a temperature range between −20° C. to room temperature, commonly −10° C. to 10° C., preferably at 0° C. For purposes of this disclosure, room temperature is herein defined as a temperature between 20° C. and 26° C., more specifically, 23° C.

The solvent matrix comprises a polar, inert solvent, such as dimethylsulfoxide, dimethylformamide, dimethylacetamide, methanol, ethanol, isopropanol, tetrahydrofuran, and acetone. The molar yields of HMF ester amphiphiles are quantitative or near so.

According to an embodiment, the vestigial —OH moiety of FDM or bHMTHF mono-ester is contacted with a sulfonating agent selected from the group consisting of p-toluenesulfonyl (tosyl), methanesulfonyl, (mesyl), ethanesulfonate (esyl), benzenesulfonate (besyl), p-bromobenzenesulfonate (brosyl), and triflouromethanesulfonic anhydride (triflate) and a sulfonate ester moiety is formed where the —OH moiety once existed. The sulfonating agent is typically trifluoromethanesulfonic anhydride. These compounds of FDM or bHMTHF mono-esters having a sulfonate ester moiety as a side chain and an additional carbon side chain of 8-26 carbons are novel compounds and can be easily isolated from the reaction.

The precursors to this reaction, FDM or THF (or bHMTHF) mono-esters, are made by contacting a FDM or THF (or bHMTHF) with a fatty acid ester in the presence of a lipase enzyme. In some embodiments, this lipase enzyme is a Candida Antarctica B lipase (CAB).

The reaction requires a nucleophilic base to furnish high yields, such as dimethylaminopyridine, imidazole, and pyrazole, but preferably pyridine, owing to its facility of removal.

The reaction is conducted in an inert organic solvent with a high vapor pressure, such as chloroform, tetrahydrofuran, acetone, benzene, diethyl ether, but preferably methylene chloride

The reaction is conducted at temperatures between −20° C. and room temperature, typically between −10° C. and 10° C., but preferably at about 0° C.

The molar yields of FDM or bHMTHF triflate esters is quantitative or near so.

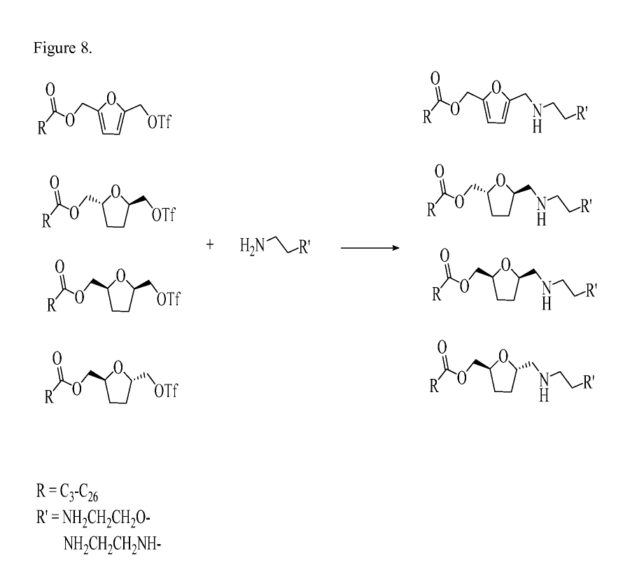

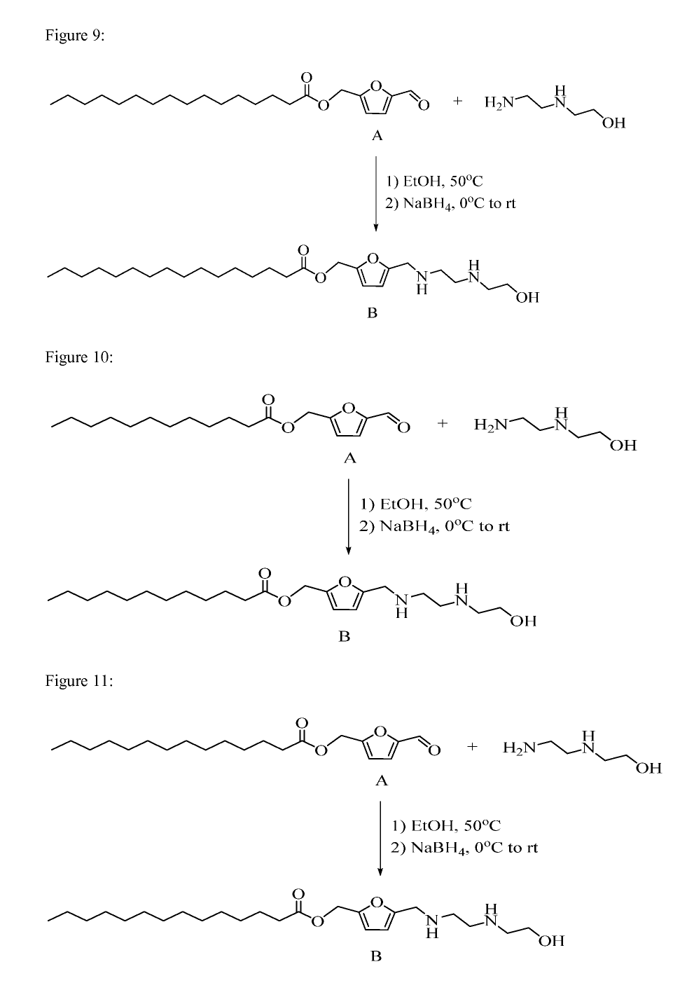

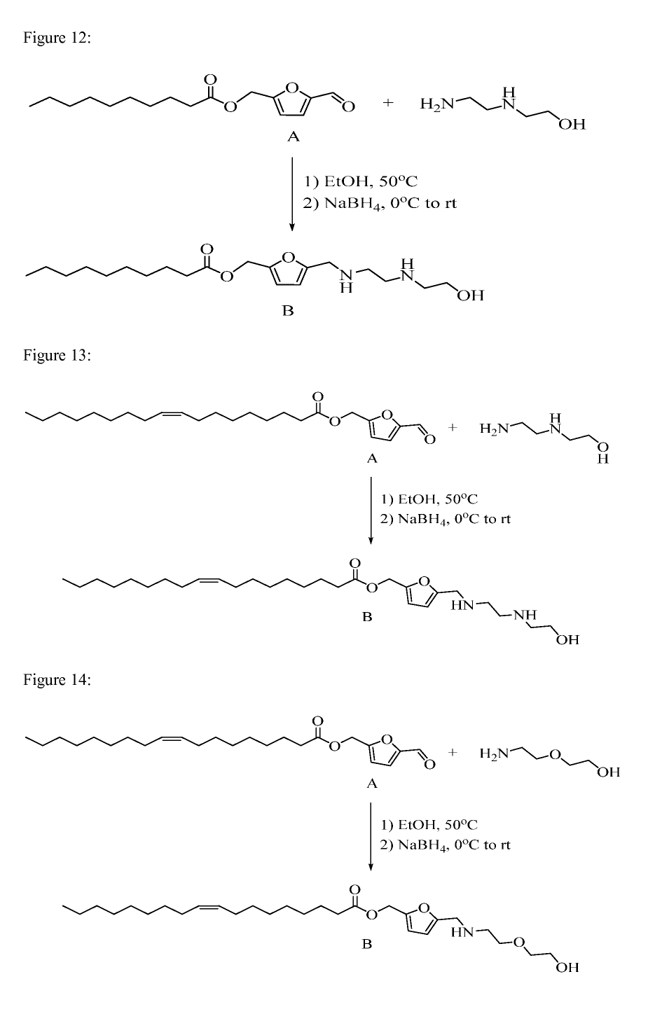

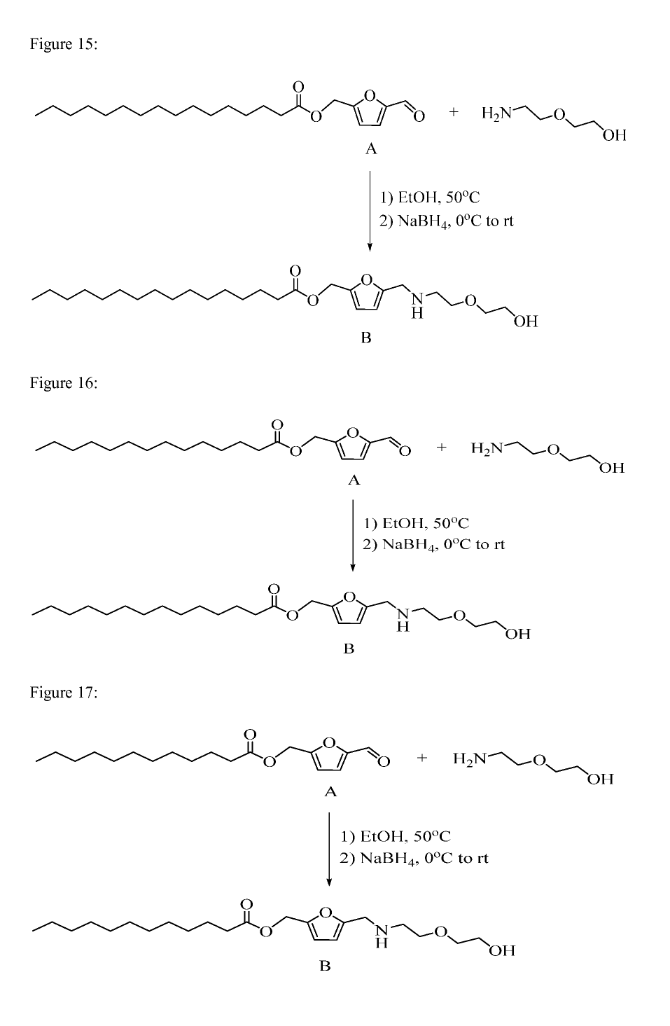

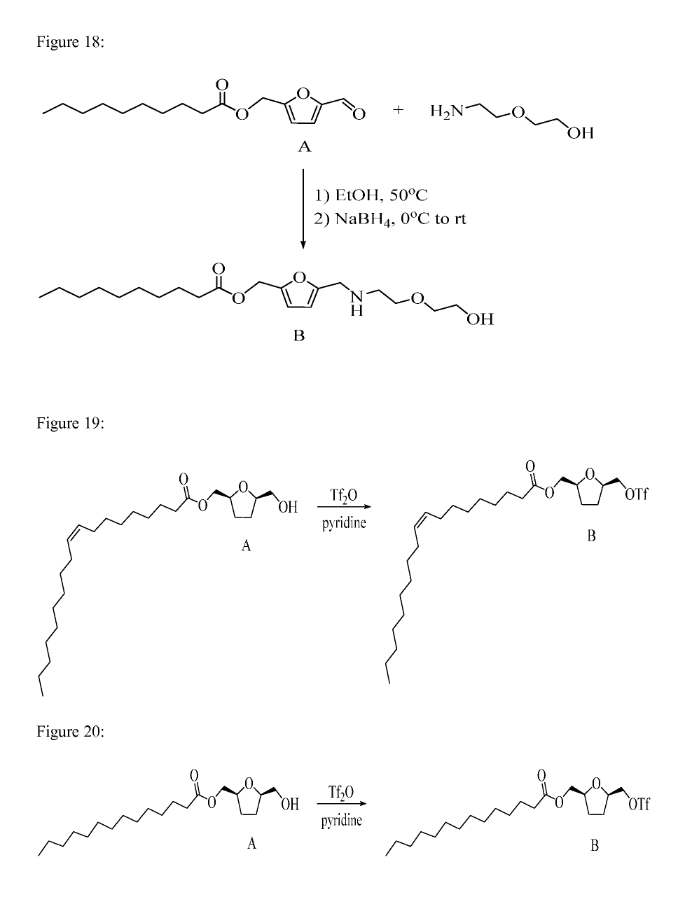

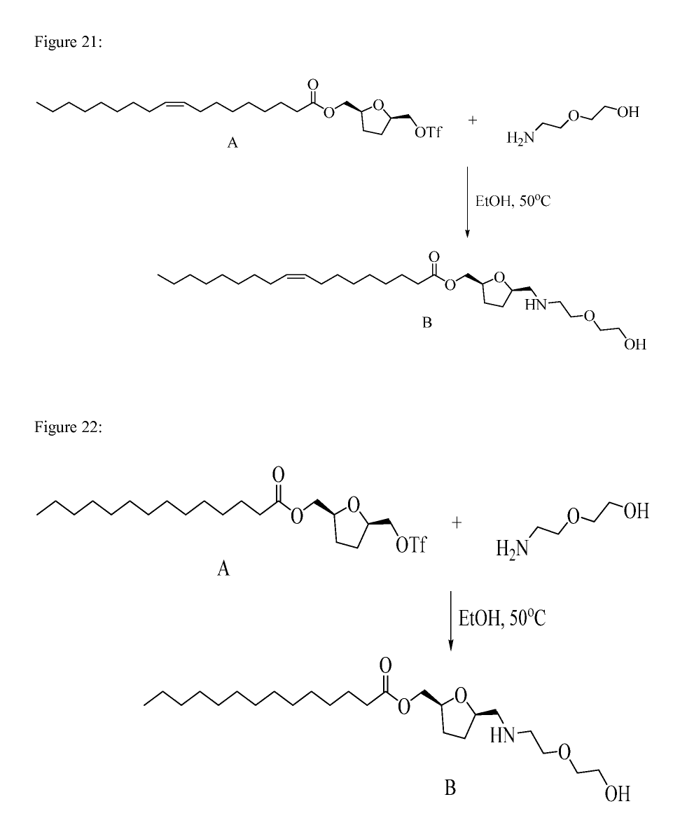

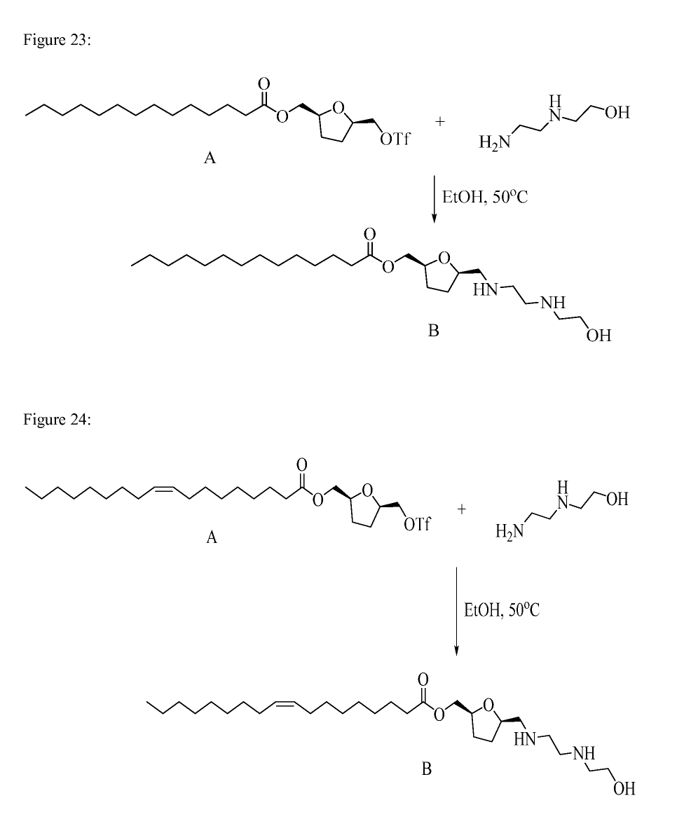

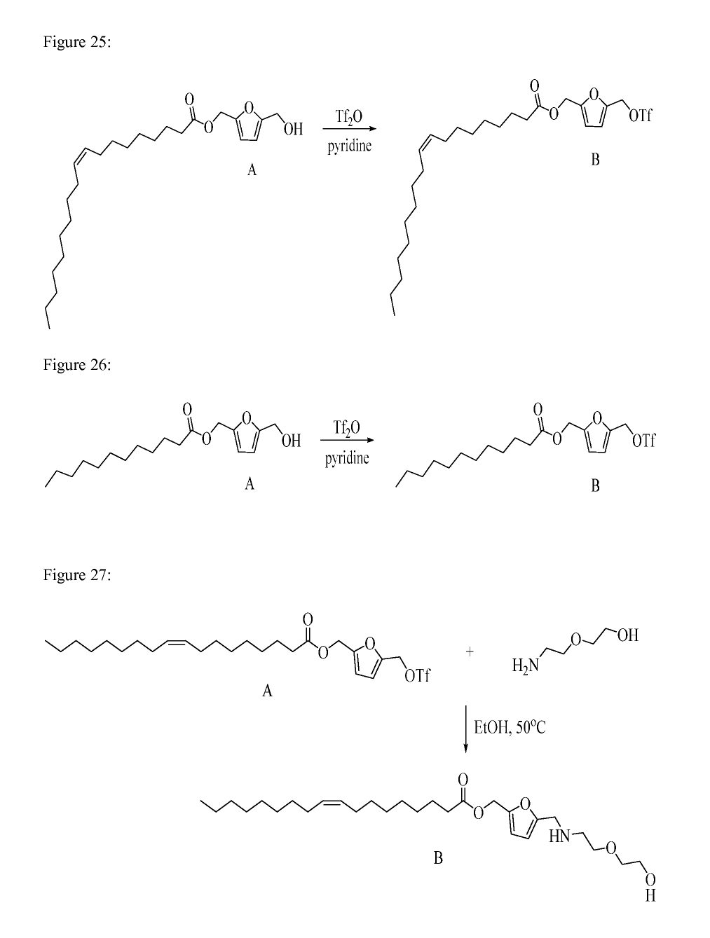

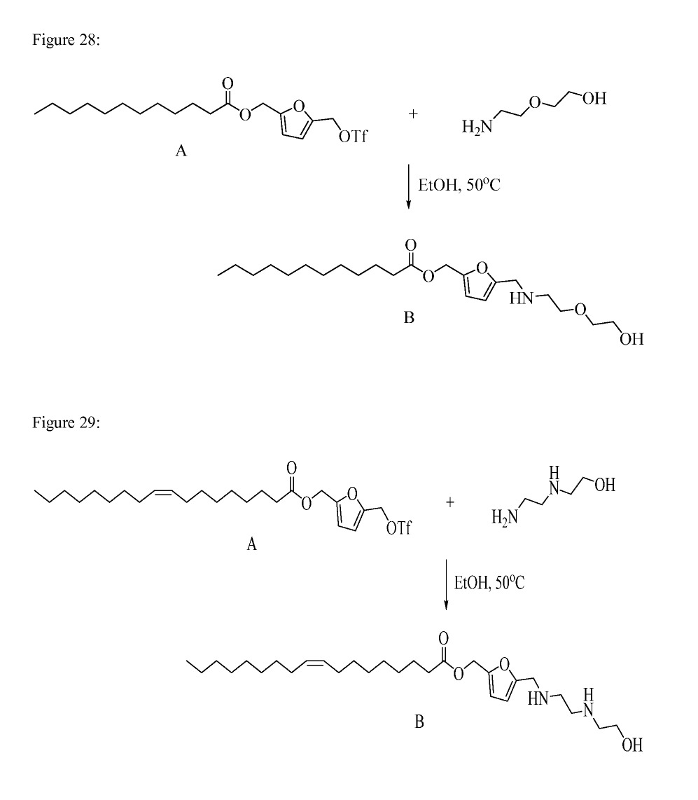

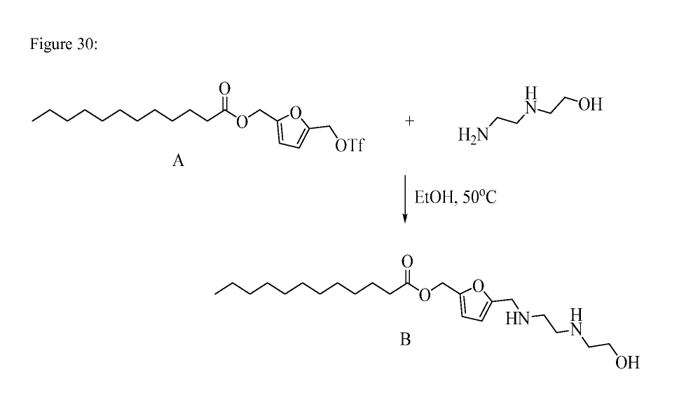

According to another embodiment, the triflated FDM or THF (bHMTHF) esters undergo nucleophilic displacement reactions with a hydrophilic amino reactant in an inert polar solvent, producing the targeted FDM or bHMTHF ester non-ionic amphiphiles (FIG. 8).

The hydrophilic amine consists of a terminal amino moiety (NH₂CH₂CH₂O—, NH₂CH₂CH₂NH—) with sufficient internal oxygen, nitrogen atoms to effectuate water solubility.

The nucleophilic substitution is conducted in an inert, polar solvent with a dielectric constant (∈>20), such as dimethylsulfoxide, dimethylformamide, dimethylacetamide, N-methylpyrrolidone, acetonitrile, methanol, ethanol, and acetone.

The reaction temperature is between 30° C. and 100° C., typically 40° C. and 80° C., preferably at about 50° C.

The molar yields of ampathic FDM or bHMTHF esters are greater than about 50%, commonly 55-95%, preferably greater than 85%.

### Examples

The following examples are furnished as demonstrative of the diverse aspects of the present disclosure, with the recognition that altering parameters and conditions, for example by change of temperature, time and reagent amounts, and particular starting species and catalysts and amounts thereof, can affect and extend the full practice of the invention beyond the limits of the examples presented.

The following examples refer to HMF, FDM, and bHMTHFs and limited fatty acids for reasons of facility, however, the scope of the invention is not relegated to those specific embodiments that inserts as other more common or commercially available fatty acid species. Examples 1-5 are examples of enzymatic synthesis of hydroxymethylfurfural (HMF) fatty acid esters. Examples 6-11 are examples of enzymatic synthesis of fatty acid esters derivatives of THF-diols, which are novel compositions of matter. Examples 12-13 are examples of enzymatic synthesis of fatty acid esters derivatives of FDM, which are novel compositions of matter. Examples 14-18 are examples of synthesis of HMF-FA-AEEA amphiphiles. Examples 19-23 are examples of synthesis of HMF-FA-AEE amphiphiles. Examples 24-29 are examples of synthesis of bHMTHF-based amphiphiles. For simplicity, in each of these examples, the preponderant cis-substituted tetrahydrofuran derivative will be underscored, with the understanding that the reagents are comprised of a 9:1 cis/trans stereoisomerism. Examples 26-29 are examples where the THF-triflate-fatty acid esters represent novel compositions of matter. Examples 30-35 are examples of synthesis of FDM-based amphiphiles. Examples 32-35 show FDM-fatty acid ester-triflates that are novel compositions of matter.

### Example 1: Preparation of HMF/Oleic Mono-Esters (80-5676 LW, 85-5676 LW)

Sample of HMF was obtained from Industrial Chemicals group of ADM Research. Technical-grade oleic acid was purchased from Sigma-Aldrich (St. Louis, Mo.). Lipozyme 435 (granular immobilized lipase enzyme from Candida Antarctica B Lipase) was obtained from Novozymes (Pagsvaerd, Denmark). A reaction mixture was comprised of 225.6 g (0.8 mol) of oleic acid, 35.8 g of enzyme, and 132.1 g (1 mol) HMF. The mixture was heated to 70° C. under vacuum at 5 torr and agitated at 400 rpm. The reaction achieved 0.50% residual free fatty acid after 3 h. A sample of mixture was taken at 3 h and analyzed on a TLC plate. A Whatman Partisil K6, 5×10 cm, silica gel 60 Å TLC plate with a thickness of 250 μm was used. A mixture of hexane/ethyl ether=1/1 was used to develop the TLC plate. The developed TLC plate was sprayed with 5% sulfuric acid in methanol and then placed in a 110° C. oven for darkening of spots on the plate. TLC analysis of the reaction mixture showed that the reaction was completed with almost all oleic acid disappeared. Mostly mono-esters were visible on the TLC plate with a minor amount of un-reacted HMF also present. The reaction mixture was filtered over a Whatman #40 filter paper to remove the enzyme. Short-path distillation was used to purify mono-esters and to remove residual fatty acids and any un-reacted HMF material. 115 g of the reaction mixture was processed at 100° C., 230 rpm rotor speed, and ˜0.04 mb vacuum. The flow rate was set at 6 mL/min. However no material was distilled at this temperature. Temperature was then increased to 112.5° C. which was sufficient to remove unreacted materials. The retentate fraction was 97 g and had 1.7% FFA remaining. TLC plating showed the fraction was exclusively mono-esters with the remaining FFA. The distillate fraction was 4 g of unreacted HMF and FFA. TLC plate showed that no mono-esters were present in the distillate fraction.

### Example 2: Preparation HMF/Lauric Mono-Esters (90-5676 LW)

Laurie acid was also reacted with HMF. The lauric acid was sourced from Tokyo Chemical Industries (TCI). The reaction mixture was comprised of 200.3 g (1 mol) of lauric acid, 21.9 g of enzyme, and 132.1 g (1 mol) HMF. The mixture was heated to 60° C. under vacuum at 5 torr and agitated 400 rpm. The reaction reached 6.3% residual FFA after 21 h. The reaction mixture was filtered over a Whatman #40 filter paper to remove the enzyme. TLC analysis of the reaction mixture showed that the reaction was completed with small amount of FFA remaining. The filtered mixture (120 g) was then processed by short-path distillation to purify mono-esters and to remove residual fatty acids. This distillation was performed at 125° C., 230 rpm rotor speed, and 0.032 mb vacuum. The flow rate was set at 8 mL/min. At this temperature, 70.9 g of purified mono-esters was recovered and a very small amount of residual unreacted/FFA was distilled off. The temperature was thought to be too high so another distillation was run and temperature was lowered to 100° C. The reaction progressed successfully with 42.5 g purified mono-esters and 6.6 g residual FFA material. Both purified mono-esters fractions, distilled at 125° C. and 100° C., were retained for further testing.

### Example 3: Preparation HMF/Capric Mono-Esters (91-5676 LW)

Another reaction was performed using capric acid to react with HMF. The capric acid (>98% purity) was sourced from Tokyo Chemical Industries (TCI). The reaction mixture was comprised of 113.7 g of capric acid, 20.1 g of enzyme, and 87.2 g HMF. The mixture was heated to 60° C. under vacuum at 5 torr and agitated 400 rpm. The reaction reached 7.5% residual FFA after 22 h. The reaction mixture was filtered over a Whatman #40 filter paper to remove the enzyme. TLC analysis of the reaction mixture showed that the reaction was completed with small amount of FFA and unreacted HMF material remaining. The filtered mixture (120 g) was then processed by short-path distillation to purify mono-esters and to remove residual fatty acids. The distillation temperature was lowered due to the greater volatility of the short-chain FA. This distillation was performed at 95° C., 230 rpm rotor speed, and 0.035 mb vacuum. The flow rate was set at 8 mL/min. 80.5 g of purified mono-esters was recovered and 17.6 g of residual, unreacted HMF and FFA was distilled off.

### Example 4: Preparation of HMF/Palmitic Mono-Esters (93-5676 LW)

A further reaction was performed using palmitic acid with HMF. The palmitic acid was 95% pure and sourced from Sigma-Aldrich (St. Louis, Mo.). The reaction mixture was comprised of 169.2 g of palmitic acid, 25.6 g of enzyme, and 87.2 g HMF. The mixture was heated to 70° C. under vacuum at 5 torr and agitated at 400 rpm. The reaction reached 6.6% residual FFA after 23 h. The reaction mixture was filtered over a Whatman #40 filter paper to remove the enzyme. TLC analysis of the reaction mixture showed that the reaction was completed with small amount of FFA and unreacted HMF remaining. The filtered mixture (200 g) was then processed by short-path distillation to purify mono-esters and to remove residual fatty acids. This distillation was performed at 135° C., 230 rpm rotor speed, and 0.035 mb vacuum. The flow rate was set at 8 mL/min. 86.5 g of purified mono-esters was recovered and 13.8 g of residual unreacted HMF and FFA was distilled off.

### Example 5: Preparation of HMF/Myristic Mono-Esters (109-5676 LW)

Myristic acid was reacted with HMF. The myristic acid was 95% pure and sourced from Sigma-Aldrich (St. Louis, Mo.). The reaction mixture was comprised of 228.4 g of myristic acid, 36.1 g of enzyme, and 132.1 g HMF. The mixture was heated to 65° C. under vacuum at 5 torr and agitated at 400 rpm. The reaction reached 3.4% residual FFA after 23 hrs. The reaction mixture was filtered over a Whatman #40 filter paper to remove the enzyme. TLC analysis of the reaction mixture showed that the reaction was completed with small amount of FFA/unreacted HMF remaining. The filtered mixture (200 g) was then processed by short-path distillation to purify mono-esters and to remove residual fatty acids. This distillation was performed at 135° C., 230 rpm rotor speed, and 0.035 mb vacuum. The flow rate was set at 8 mL/min. 110 g of purified mono-esters was recovered having 0.67% FFA remaining. The distillate fraction was 16.1 g of residual, unreacted HMF and concentrated FFA.

### Example 6: Preparation of THF/Oleic Mono-Esters (65-5676 LW)

Sample of Tetrahydrofuran diols (THF) was obtained from Industrial Chemicals group of ADM Research. Technical-grade oleic acid was purchased from Sigma-Aldrich (St. Louis, Mo.). Lipozyme 435 (granular immobilized lipase enzyme from Candida Antarctica B Lipase) was obtained from Novozymes (Pagsvaerd, Denmark). A reaction mixture was comprised of 70.5 g (1 mol) of oleic acid, 10.3 g of enzyme, and 33 g (1 mol) THF. The mixture was heated to 80° C. under vacuum at 5 torr and agitated at 400 rpm. The reaction achieved 0.2% residual free fatty acid after 1.5 hr. A sample of mixture was taken at 2 hr and analyzed on a TLC plate. A Whatman Partisil K6, 5×10 cm, silica gel 60 Å TLC plate with a thickness of 250 μm was used. A mixture of hexane/ethyl ether=1/1 was used to develop the TLC plate. The developed TLC plate was sprayed with 5% sulfuric acid in methanol and then placed in a 110° C. oven for darkening of spots on the plate. TLC analysis of the reaction mixture showed that the reaction was completed with almost all oleic acid disappeared. Mostly mono-esters were visible on the TLC plate with a minor amount of di-esters and un-reacted THF also present. The reaction mixture was filtered over a Whatman #40 filter paper to remove the enzyme and recover reaction mixture.

### Example 7: Preparation of THF/Oleic Mono-Esters (65-5676 LW)

A further reaction was performed using 2 mol/1 mol ratio of oleic: THF. The reaction was carried out as in Example 6 using 112.8 g oleic acid, 12.8 g Lipozyme 435 enzyme, and 26.4 g THF. The reaction reached 4.0% residual FFA after 4 hr. The reaction was further carried out overnight for a total of 20 hr reaction time resulting in 1.5% residual FFA. TLC plating showed a large fraction of di-esters remaining.

### Example 8: Preparation of THF/Oleic Mono-Esters (66-5676 LW)

Another reaction was performed using 1:1 mol ration of oleic: THF as in example 6 except reaction temperature was increased to 60° C. in an attempt to further push the reaction toward making mono-esters. Oleic acid (70.5 g) was heated to 60° C. before adding 10.3 g Lipozyme 435 enzyme. THF (33 g) was then added. The reaction was completed after 2 h with 0.48% residual FFA remaining. TLC plating showed mostly mono-esters present.

### Example 9: Preparation of THF/Oleic Mono-Esters (68-5676 LW)

Yet another reaction was performed using 1:0.8 mol ratio of oleic: THF to promote mono-esters formation. A reaction mixture was comprised of 75 g (1 mol) of oleic acid, 11.9 g of Lipozyme 435 enzyme, and 44 g (1 mol) THF. The mixture was heated to 80° C. under vacuum at 5 torr and agitated at 350 rpm. The reaction achieved 0.53% residual free fatty acid after 1 hr. A sample of mixture was taken at 1 hr and analyzed on a TLC plate. TLC plating showed mostly mono-esters present but some di-esters also remaining.

### Example 10: Preparation of THF/Oleic Mono-Esters (72-5676 LW, 84-5676 LW)

A further reaction using Lipozyme RMIM (granular immobilized lipase enzyme from Candida Antarctica B Lipase) obtained from Novozymes (Pagsvaerd, Denmark) was tried to further promote mono-esters formation. A reaction mixture was comprised of 75 g (0.8 mol) of oleic acid, 11.9 g of enzyme, and 44 g (1 mol) THF. The mixture was heated to 55° C. under vacuum at 5 torr and agitated at 350 rpm. The reaction achieved 11.8% residual free fatty acid after 1 hr. and only further reduced to 11.6% after 2 hr. A sample of mixture was taken at 2 hr and analyzed on a TLC plate. TLC plating was performed as in previous examples. TLC analysis of the reaction mixture showed that the reaction was completed with almost all oleic acid disappeared. Mostly mono-esters were visible on the TLC plate with a minor amount of di-esters and un-reacted THF also present. The reaction mixture was filtered over a Whatman #40 filter paper to remove the enzyme. Short-path distillation was used to purify mono-esters and to remove residual fatty acids and any un-reacted THF material. The reaction mixture (110 g) was processed at 210° C., 230 rpm rotor speed, and ˜0.045 mb vacuum with 6 mL/min flow rate. These conditions were sufficient to remove the di-esters fraction (39 g). The retentate fraction was 62.3 g and had 0.9% FFA remaining. This retentate fraction comprised of mono-esters and residual FFA was then further distilled at 155° C. to further purify the mono-esters. This retentate fraction (33.4 g) had 0.2% FFA remaining. TLC plating showed the fraction was exclusively mono-esters with the remaining FFA. The distillate fraction was 10.2 g of unreacted THF and FFA. TLC plate showed that no mono-esters were present in the distillate fraction.

### Example 11: Preparation of THF/Myristic Mono-Esters (191-5676 LW)

Myristic acid was obtained from Sigma-Aldrich (St. Louis, Mo.). The reaction mixture was comprised of 114.1 g myristic acid, 18 g of enzyme, and 66.1 g THF. The mixture was heated to 65° C. under vacuum at 5 torr and agitated 400 rpm. The reaction was completed after 2 h with 0.3% residual FFA remaining. TLC plating showed mono-esters present, but some di-esters still remaining. The filtered reaction mixture was processed by short-path distillation to purify mono-esters and separate di-esters and residual FFA. The distillation was performed at 210° C. to separate the di-esters. The mono-esters/FFA fraction was further processed at 125° C. to separate the residual FFA. 40 g of purified mono-esters was recovered having 0.45% FFA remaining.

### Example 12: Preparation of FDM/Oleic Mono-Esters (130-5676 LW)

Sample of Furan 2,5 Diemethanol diols (FDM) was obtained from Industrial Chemicals group of ADM Research. Technical-grade oleic acid was purchased from Sigma-Aldrich (St. Louis, Mo.). Lipozyme 435 (granular immobilized lipase enzyme from Candida Antarctica B Lipase) was obtained from Novozymes (Pagsvaerd, Denmark). A reaction mixture was comprised of 113 g (1 mol) of oleic acid, 18 g of enzyme, and 66 g (1 mol) FDM. The mixture was heated to 70° C. under vacuum at 5 torr and agitated at 400 rpm. The reaction achieved 1.0% residual free fatty acid after 3 hr. A sample of mixture was taken at 3 hr and analyzed on a TLC plate. A Whatman Partisil K6, 5×10 cm, silica gel 60 Å TLC plate with a thickness of 250 μm was used. A mixture of hexane/ethyl ether=1/1 was used to develop the TLC plate. The developed TLC plate was sprayed with 5% sulfuric acid in methanol and then placed in a 110° C. oven for darkening of spots on the plate. TLC analysis of the reaction mixture showed that the reaction was completed with almost all oleic acid disappeared. Mostly mono-esters were visible on the TLC plate with a minor amount of di-esters and un-reacted THF also present. The reaction mixture was filtered over a Whatman #40 filter paper to remove the enzyme. The reaction was repeated to generate more material following the above conditions. Short-path distillation was used to purify the mono-esters and to remove the residual fatty acids and any un-reacted FDM material. 250 g of the reaction mixture was processed at 210° C. to remove the di-esters fraction (101.7 g). The remaining mon-esters/FFA fraction was then processed at 155° C. to purify the mono-esters. The retentate fraction was exclusively mono-esters with 0.5% FFA remaining. The distillate fraction was 26 g of un-reacted FDM and FFA material.

### Example 13: Preparation of FDM/Lauric Mono-Esters (190-5676 LW)

Lauric acid was sourced from Tokyo Chemical Industries (TCI). The reaction mixture was comprised of 100 g (1 mol) of lauric acid, 18 g enzyme, and 66 g FDM (1 mol). The reaction was performed as in example 12. The reaction achieved 0.6% FFA after 2 hr. The reaction mixture was filtered before short-path distillation to purify the mono-esters. The distillation was performed as in example 12. 39 g of di-esters fraction and 40 g of mono-esters fraction was recovered. The mono-esters fraction had 0.2% FFA remaining.

### Example 14: Synthesis of (5-(((2-((2-hydroxyethyl)amino)ethyl)amino) methyl)furan-2-yl)methyl palmitate, B

Experimental: A single neck 10 mL round bottomed flask was charged with 100 mg of HMF-palmitate A (0.274 mmol) and 5 mL of absolute ethanol. While stirring, 28.5 mg of 2-((2-aminoethyl)amino)ethanol (0.274 mmol) dissolved in 2 mL of absolute ethanol was added dropwise over 5 minutes. The flask was then outfitted with a reflux condenser with an attached argon line, and, while vigorously stirring and under argon, heated to 50° C. and the reaction continued at this temperature overnight. After this time, an aliquot was removed and analyzed by ¹H NMR, disclosing the absence of the signature aldehyde signal of A. The solution was cooled to room temperature, then immersed in a brine/ice bath until the internal temperature read 0° C., and charged with 11.5 mg of sodium borohydride (0.302 mmol) in two portions 5 minutes apart. After addition, the ice bath was removed and solution stirred for 2 h at ambient temperature. ¹H NMR analysis of an aliquot after this time manifested no characteristic imine signal of the intermediate, adducing that full reduction to the amine had occurred. A 1 mL volume of a 1 M HCl solution was then added to quench any remaining reducing agent. The solution was then poured onto a prefabricated column packed with activated, Brockmann I neutral alumina employing absolute ethanol as the eluent, and 91 mg of B was isolated as a colorless, loose oil (73% of theoretical) after concentration. ¹H NMR (400 MHz, anhydrous CDCl₃) δ (ppm) 6.41 (d, J=7.2 Hz, 1H), 6.19 (d, J=7.2 Hz, 1H), 5.21 (s, 2H), 3.77 (s, 2H), 3.65 (m, 1H), 3.39 (t, J=8.0 Hz, 2H), 3.08 (m, 2H), 2.69 (t, J=7.9 Hz, 2H), 2.48 (m, 4H), 2.21 (t, J=7.4 Hz, 2H), 1.71 (m, 2H), 1.30-1.27 (m, 24H), 0.91 (t, J=7.0 Hz, 3H); ¹³C NMR (100 MHz, anhydrous CDCl₃) δ (ppm) 171.2, 149.4, 136.2, 108.1, 106.9, 62.1, 60.9, 51.1, 50.3, 49.1, 47.7, 35.8, 32.2, 30.5, 30.1, 29.8, 29.6, 29.4, 29.3, 29.1, 28.8, 28.6, 28.2, 27.6, 27.3, 12.8.

### Example 15: Synthesis of (5-(((2-((2-hydroxyethyl)amino)ethyl)amino) methyl)furan-2-yl)methyl laurate, B

Experimental: A single neck 10 mL round bottomed flask was charged with 100 mg of HMF-laurate A (0.324 mmol) and 5 mL of absolute ethanol. While stirring, 33.8 mg of 2-((2-aminoethyl)amino)ethanol (0.324 mmol) dissolved in 2 mL of absolute ethanol was added dropwise over 5 minutes. The flask was then outfitted with a reflux condenser with an attached argon line, and, while vigorously stirring and under argon, heated to 50° C. and the reaction continued at this temperature overnight. After this time, an aliquot was removed and analyzed by ¹H NMR, disclosing the absence of the signature aldehyde signal of A. The solution was cooled to room temperature, then immersed in a brine/ice bath until the internal temperature read 0° C., and charged with 13.5 mg of sodium borohydride (0.357 mmol) in two portions 5 minutes apart. After addition, the ice bath was removed and solution stirred for 2 h at ambient temperature. ¹H NMR analysis of an aliquot after this time manifested no characteristic imine signal of the intermediate, adducing that full reduction to the amine had occurred. A 1 mL volume of a 1 M HCl solution was then added to quench any remaining reducing agent. The solution was then poured onto a prefabricated column packed with activated, Brockmann I neutral alumina employing absolute ethanol as the eluent, and 97 mg of B was isolated as pale yellow, loose oil (75% of theoretical) after concentration. ¹H NMR (400 MHz, anhydrous CDCl₃) δ (ppm) 6.40 (d, J=7.2 Hz, 1H), 6.21 (d, J=7.2 Hz, 1H), 5.19 (s, 2H), 3.78 (s, 2H), 3.66 (m, 1H), 3.39 (m, 2H), 3.06 (m, 2H), 2.69 (t, J=7.9 Hz, 2H), 2.48 (m, 4H), 2.21 (t, J=7.4 Hz, 2H), 1.71 (m, 2H), 1.30-1.27 (m, 16H), 0.91 (t, J=7.0 Hz, 3H); ¹³C NMR (100 MHz, anhydrous CDCl₃) δ (ppm) 171.2, 149.4, 136.2, 108.1, 106.9, 62.1, 60.9, 51.1, 50.3, 49.1, 47.7, 35.8, 32.2, 30.3, 29.6, 29.2, 28.9, 28.6, 28.2, 27.6, 27.1, 12.6.

### Example 16: Synthesis of (5-(((2-((2-hydroxyethyl)amino)ethyl)amino) methyl)furan-2-yl)methyl myristate, B

Experimental: A single neck 10 mL round bottomed flask was charged with 100 mg of HMF-myristate A (0.297 mmol) and 5 mL of absolute ethanol. While stirring, 30.9 mg of 2-((2-aminoethyl)amino)ethanol (0.297 mmol) dissolved in 2 mL of absolute ethanol was added dropwise over 5 minutes. The flask was then outfitted with a reflux condenser with an attached argon line, and, while vigorously stirring and under argon, heated to 50° C. and the reaction continued at this temperature overnight. After this time, an aliquot was removed and analyzed by ¹H NMR, disclosing the absence of the signature aldehyde signal of A. The solution was cooled to room temperature, then immersed in a brine/ice bath until the internal temperature read 0° C., and charged with 12.4 mg of sodium borohydride (0.327 mmol) in two portions 5 minutes apart. After addition, the ice bath was removed and solution stirred for 2 h at ambient temperature. ¹H NMR analysis of an aliquot after this time manifested no characteristic imine signal of the intermediate, adducing that full reduction to the amine had occurred. A 1 mL volume of a 1 M HCl solution was then added to quench any remaining reducing agent. The solution was then poured onto a prefabricated column packed with activated, Brockmann I neutral alumina employing absolute ethanol as the eluent, and 88 mg of B was isolated as a colorless, loose oil (70% of theoretical) after concentration. ¹H NMR (400 MHz, anhydrous CDCl₃) δ (ppm) 6.41 (d, J=7.2 Hz, 1H), 6.19 (d, J=7.2 Hz, 1H), 5.21 (s, 2H), 3.77 (s, 2H), 3.65 (m, 1H), 3.39 (m, 2H), 3.08 (m, 2H), 2.69 (t, J=7.9 Hz, 2H), 2.48 (m, 4H), 2.21 (t, J=7.4 Hz, 2H), 1.71 (m, 2H), 1.30-1.27 (m, 20H), 0.91 (t, J=7.0 Hz, 3H); ¹³C NMR (100 MHz, anhydrous CDCl₃) δ (ppm) 171.2, 149.4, 136.2, 108.1, 106.9, 62.1, 60.9, 51.1, 50.3, 49.1, 47.7, 35.8, 32.2, 30.5, 30.1, 29.8, 29.6, 29.4, 29.3, 28.9, 28.4, 27.6, 27.3, 12.7.

### Example 17: Synthesis of (5-(((2-((2-hydroxyethyl)amino)ethyl)amino) methyl)furan-2-yl)methyl caprate, B

Experimental: A single neck 10 mL round bottomed flask was charged with 100 mg of HMF-caprate A (0.357 mmol) and 5 mL of absolute ethanol. While stirring, 37.1 mg of 2-((2-aminoethyl)amino)ethanol (0.324 mmol) dissolved in 2 mL of absolute ethanol was added dropwise over 5 minutes. The flask was then outfitted with a reflux condenser with an attached argon line, and, while vigorously stirring and under argon, heated to 50° C. and the reaction continued at this temperature overnight. After this time, an aliquot was removed and analyzed by ¹H NMR, disclosing the absence of the signature aldehyde signal of A. The solution was cooled to room temperature, then immersed in an brine/ice bath until the internal temperature read 0° C., and charged with 14.8 mg of sodium borohydride (0.392 mmol) in two portions 5 minutes apart. After addition, the ice bath was removed and solution stirred for 2 h at ambient temperature. ¹H NMR analysis of an aliquot after this time manifested no characteristic imine signal of the intermediate, adducing that full reduction to the amine had occurred. A 1 mL volume of a 1 M HCl solution was then added to quench any remaining reducing agent. The solution was then poured onto a prefabricated column packed with activated, Brockmann I neutral alumina employing absolute ethanol as the eluent, and 102 mg of B was isolated as a colorless semi-solid (78% of theoretical) after concentration. ¹H NMR (400 MHz, anhydrous CDCl₃) δ (ppm) 6.40 (d, J=7.2 Hz, 1H), 6.21 (d, J=7.2 Hz, 1H), 5.19 (s, 2H), 3.78 (s, 2H), 3.66 (m, 1H), 3.39 (m, 2H), 3.06 (m, 2H), 2.69 (t, J=7.9 Hz, 2H), 2.48 (m, 4H), 2.21 (t, J=7.4 Hz, 2H), 1.71 (m, 2H), 1.30-1.27 (m, 12H), 0.91 (t, J=7.0 Hz, 3H); ¹³C NMR (100 MHz, anhydrous CDCl₃) δ (ppm) 171.2, 149.4, 136.2, 108.1, 106.9, 62.1, 60.9, 51.1, 50.3, 49.1, 47.7, 35.8, 32.2, 30.3, 29.6, 29.1, 28.3, 27.6, 27.1, 12.9.

### Example 18: Synthesis of (5-(((2-((2-hydroxyethyl)amino)ethyl)amino) methyl)furan-2-yl)-methyl oleate, B

Experimental: A single neck 10 mL round bottomed flask was charged with 100 mg of HMF-oleate A (0.256 mmol) and 5 mL of absolute ethanol. While stirring, 26.7 mg of 2-((2-aminoethyl)amino)ethanol (0.256 mmol) dissolved in 2 mL of absolute ethanol was added dropwise over 5 minutes. The flask was then outfitted with a reflux condenser with an attached argon line, and, while vigorously stirring and under argon, heated to 50° C. and the reaction continued at this temperature overnight. After this time, an aliquot was removed and analyzed by ¹H NMR, disclosing the absence of the signature aldehyde signal of A. The solution was cooled to room temperature, then immersed in an brine/ice bath until the internal temperature read 0° C., and charged with 11 mg of sodium borohydride (0.282 mmol)) in two portions 5 minutes apart. After addition, the ice bath was removed and solution stirred for 2 h at ambient temperature. ¹H NMR analysis of an aliquot after this time manifested no characteristic imine signal of the intermediate, adducing that full reduction to the amine had occurred. A 1 mL volume of a 1 M HCl solution was then added to quench any remaining reducing agent. The solution was then poured onto a prefabricated column packed with activated, Brockmann I neutral alumina employing absolute ethanol as the eluent, and 74 mg of B was isolated as a colorless, loose oil (60% of theoretical) after concentration. ¹H NMR (400 MHz, anhydrous CDCl₃) δ (ppm) 6.41 (d, J=7.2 Hz, 1H), 6.19 (d, J=7.2 Hz, 1H), 5.47 (m, 2H), 5.21 (s, 2H), 3.77 (s, 2H), 3.65 (m, 1H), 3.39 (m, 2H), 3.08 (m, 2H), 2.69 (t, J=7.9 Hz, 2H), 2.48 (m, 4H), 2.21-2.19 (m, 6H), 1.71 (m, 2H), 1.30-1.27 (m, 20H), 0.91 (t, J=7.0 Hz, 3H); ¹³C NMR (100 MHz, anhydrous CDCl₃) δ (ppm) 171.2, 149.4, 136.2, 130.8, 130.6, 108.1, 106.9, 62.1, 60.9, 51.1, 50.3, 49.1, 47.7, 35.8, 32.2, 30.5, 30.1, 29.8, 29.6, 29.4, 29.3, 29.1, 28.8, 28.6, 28.2, 27.6, 27.3, 12.8.

### Example 19: Synthesis of (5-(((2-(2-hydroxyethoxy)ethyl)amino) methyl)furan-2-yl)-methyl oleate, B

Experimental: A single neck 10 mL round bottomed flask was charged with 100 mg of HMF-oleate A (0.256 mmol) and 5 mL of absolute ethanol. While stirring, 26.9 mg of 2 2-(2-aminoethoxy)ethanol (0.256 mmol) dissolved in 2 mL of absolute ethanol was added dropwise over 5 minutes. The flask was then outfitted with a reflux condenser with an attached argon line, and, while vigorously stirring and under argon, heated to 50° C. and the reaction continued at this temperature overnight. After this time, an aliquot was removed and analyzed by ¹H NMR, disclosing the absence of the signature aldehyde signal of A. The solution was cooled to room temperature, then immersed in a brine/ice bath until the internal temperature read 0° C., and charged with 11 mg of sodium borohydride (0.282 mmol) in two portions 5 minutes apart. After addition, the ice bath was removed and solution stirred for 2 h at ambient temperature. ¹H NMR analysis of an aliquot after this time manifested no characteristic imine signal of the intermediate, adducing that full reduction to the amine had occurred. A 1 mL volume of a 1 M HCl solution was then added to quench any remaining reducing agent. The solution was then poured onto a prefabricated column packed with activated, Brockmann I neutral alumina employing absolute ethanol as the eluent, and 79 mg of B was isolated as a light yellow, loose oil (63% of theoretical) after concentration. ¹H NMR (400 MHz, anhydrous CDCl₃) δ (ppm) 6.41 (d, J=7.2 Hz, 1H), 6.19 (d, J=7.2 Hz, 1H), 5.47 (m, 2H), 5.21 (s, 2H), 3.68-3.63 (m, 10H), 2.48 (m, 4H), 2.21-2.19 (m, 6H), 1.71 (m, 2H), 1.30-1.27 (m, 18H), 0.91 (t, J=7.0 Hz, 3H); ¹³C NMR (100 MHz, anhydrous CDCl₃) δ (ppm) 171.2, 149.4, 136.2, 130.8, 130.6, 108.1, 106.9, 71.1, 70.5, 61.3, 60.6, 51.7, 47.7, 35.8, 32.2, 30.5, 30.1, 29.8, 29.6, 29.4, 29.3, 29.1, 28.8, 28.6, 28.2, 27.6, 27.3, 12.8.

### Example 20: Synthesis of (5-(((2-(2-hydroxyethoxy)ethyl)amino) methyl)furan-2-yl)-methyl palmitate, B

Experimental: A single neck 10 mL round bottomed flask was charged with 100 mg of HMF-palmitate A (0.274 mmol) and 5 mL of absolute ethanol. While stirring, 28.8 mg of 2-(2-aminoethoxy)ethanol (0.274 mmol) dissolved in 2 mL of absolute ethanol was added dropwise over 5 minutes. The flask was then outfitted with a reflux condenser with an attached argon line, and, while vigorously stirring and under argon, heated to 50° C. and the reaction continued at this temperature overnight. After this time, an aliquot was removed and analyzed by ¹H NMR, disclosing the absence of the signature aldehyde signal of A. The solution was cooled to room temperature, then immersed in a brine/ice bath until the internal temperature read 0° C., and charged with 11.5 mg of sodium borohydride (0.302 mmol) in two portions 5 minutes apart. After addition, the ice bath was removed and solution stirred for 2 h at ambient temperature. ¹H NMR analysis of an aliquot after this time manifested no characteristic imine signal of the intermediate, adducing that full reduction to the amine had occurred. A 1 mL volume of a 1 M HCl solution was then added to quench any remaining reducing agent. The solution was then poured onto a prefabricated column packed with activated, Brockmann I neutral alumina employing absolute ethanol as the eluent, and 102 mg of B was isolated as a colorless, loose oil (81% of theoretical) after concentration. ¹H NMR (400 MHz, anhydrous CDCl₃) δ (ppm) 6.41 (d, J=7.2 Hz, 1H), 6.19 (d, J=7.2 Hz, 1H), 5.21 (s, 2H), 3.68-3.64 (m, 8H), 3.39 (m, 2H), 2.70 (m, 2H), 2.21 (t, J=7.4 Hz, 2H), 1.71 (m, 2H), 1.30-1.27 (m, 24H), 0.91 (t, J=7.0 Hz, 3H); ¹³C NMR (100 MHz, anhydrous CDCl₃) δ (ppm) 171.2, 149.4, 136.2, 108.1, 106.9, 71.1, 70.5, 61.3, 60.6, 51.7, 47.7, 35.8, 32.2, 30.5, 30.1, 29.8, 29.6, 29.4, 29.3, 29.1, 28.8, 28.6, 28.2, 27.6, 27.3, 12.8.

### Example 21: Synthesis of 5-(((2-(2-hydroxyethoxy)ethyl)amino)methyl)furan-2-yl)methyl myristate, B

Experimental: A single neck 10 mL round bottomed flask was charged with 100 mg of HMF-myristate A (0.297 mmol) and 5 mL of absolute ethanol. While stirring, 31.2 mg of 2-(2-aminoethoxy)ethanol (0.297 mmol) dissolved in 2 mL of absolute ethanol was added dropwise over 5 minutes The flask was then outfitted with a reflux condenser with an attached argon line, and, while vigorously stirring and under argon, heated to 50° C. and the reaction continued at this temperature overnight. After this time, an aliquot was removed and analyzed by ¹H NMR, disclosing the absence of the signature aldehyde signal of A. The solution was cooled to room temperature, then immersed in a brine/ice bath until the internal temperature read 0° C., and charged with 12.4 mg of sodium borohydride (0.327 mmol) in two portions 5 minutes apart. After addition, the ice bath was removed and solution stirred for 2 h at ambient temperature. ¹H NMR analysis of an aliquot after this time manifested no characteristic imine signal of the intermediate, adducing that full reduction to the amine had occurred. A 1 mL volume of a 1 M HCl solution was then added to quench any remaining reducing agent. The solution was then poured onto a prefabricated column packed with activated, Brockmann I neutral alumina employing absolute ethanol as the eluent, and 90 mg of B was isolated as a pale yellow oil (71% of theoretical) after concentration. ¹H NMR (400 MHz, anhydrous CDCl₃) δ (ppm) 6.41 (d, J=7.2 Hz, 1H), 6.19 (d, J=7.2 Hz, 1H), 5.21 (s, 2H), 3.68-3.64 (m, 8H), 3.39 (m, 2H), 2.70 (m, 2H), 2.21 (t, J=7.4 Hz, 2H), 1.71 (m, 2H), 1.30-1.27 (m, 20H), 0.91 (t, J=7.0 Hz, 3H); ¹³C NMR (100 MHz, anhydrous CDCl₃) δ (ppm) 171.2, 149.4, 136.2, 108.1, 106.9, 71.1, 70.5, 61.3, 60.6, 51.7, 47.7, 35.8, 32.2, 30.5, 30.1, 29.7, 29.4, 29.0, 28.8, 28.6, 28.2, 27.6, 27.3, 12.8.

### Example 22: Synthesis of (5-(((2-(2-hydroxyethoxy)ethyl)amino) methyl)furan-2-yl)-methyl laurate, B

Experimental: A single neck 10 mL round bottomed flask was charged with 100 mg of HMF-laurate A (0.324 mmol) and 5 mL of absolute ethanol. While stirring, 34.1 mg of 2-(2-aminoethoxy)ethanol (0.324 mmol) dissolved in 2 mL of absolute ethanol was added dropwise over 5 minutes. The flask was then outfitted with a reflux condenser with an attached argon line, and, while vigorously stirring and under argon, heated to 50° C. and the reaction continued at this temperature overnight. After this time, an aliquot was removed and analyzed by ¹H NMR, disclosing the absence of the signature aldehyde signal of A. The solution was cooled to room temperature, then immersed in a brine/ice bath until the internal temperature read 0° C., and charged with 13.5 mg of sodium borohydride (0.357 mmol) in two portions 5 minutes apart. After addition, the ice bath was removed and solution stirred for 2 h at ambient temperature. ¹H NMR analysis of an aliquot after this time manifested no characteristic imine signal of the intermediate, adducing that full reduction to the amine had occurred. A 1 mL volume of a 1 M HCl solution was then added to quench any remaining reducing agent. The solution was then poured onto a prefabricated column packed with activated, Brockmann I neutral alumina employing absolute ethanol as the eluent, and 107 mg of B was isolated as a loose, colorless oil (78% of theoretical) after concentration. ¹H NMR (400 MHz, anhydrous CDCl₃) δ (ppm) 6.41 (d, J=7.2 Hz, 1H), 6.19 (d, J=7.2 Hz, 1H), 5.21 (s, 2H), 3.68-3.64 (m, 8H), 3.39 (m, 2H), 2.70 (m, 2H), 2.21 (t, J=7.4 Hz, 2H), 1.71 (m, 2H), 1.30-1.27 (m, 16H), 0.91 (t, J=7.0 Hz, 3H); ¹³C NMR (100 MHz, anhydrous CDCl₃) δ (ppm) 171.2, 149.4, 136.2, 108.1, 106.9, 71.1, 70.5, 61.3, 60.6, 51.7, 47.7, 35.8, 32.2, 30.3, 29.7, 29.1, 28.8, 28.6, 28.2, 27.6, 27.3, 12.8.

### Example 23: Synthesis of (5-(((2-(2-hydroxyethoxy)ethyl)amino) methyl)furan-2-yl)-methyl caprate, B

Experimental: A single neck 10 mL round bottomed flask was charged with 100 mg of HMF-caprate A (0.357 mmol) and 5 mL of absolute ethanol. While stirring, 37.5 mg of 2-(2-aminoethoxy)ethanol (0.357 mmol) dissolved in 2 mL of absolute ethanol was added drop wise over 5 minutes. The flask was then outfitted with a reflux condenser with an attached argon line, and, while vigorously stirring and under argon, heated to 50° C. and the reaction continued at this temperature overnight. (The reaction at this point could have been stopped and the imine form of HMF compound could have easily been isolated.) After this time, an aliquot was removed and analyzed by ¹H NMR, disclosing the absence of the signature aldehyde signal of A. The solution was cooled to room temperature, then immersed in a brine/ice bath until the internal temperature read 0° C., and charged with 14.8 mg of sodium borohydride (0.392 mmol) in two portions 5 minutes apart. After addition, the ice bath was removed and solution stirred for 2 h at ambient temperature. ¹H NMR analysis of an aliquot after this time manifested no characteristic imine signal of the intermediate, adducing that full reduction to the amine had occurred. A 1 mL volume of a 1 M HCl solution was then added to quench any remaining reducing agent. The solution was then poured onto a prefabricated column packed with activated, Brockmann I neutral alumina employing absolute ethanol as the eluent, and 122 mg of B was isolated as a colorless semi-solid (81% of theoretical) after concentration. ¹H NMR (400 MHz, anhydrous CDCl₃) δ (ppm) 6.41 (d, J=7.2 Hz, 1H), 6.19 (d, J=7.2 Hz, 1H), 5.21 (s, 2H), 3.68-3.64 (m, 8H), 3.39 (m, 2H), 2.70 (m, 2H), 2.21 (t, J=7.4 Hz, 2H), 1.71 (m, 2H), 1.30-1.27 (m, 12H), 0.91 (t, J=7.0 Hz, 3H); ¹³C NMR (100 MHz, anhydrous CDCl₃) δ (ppm) 171.2, 149.4, 136.2, 108.1, 106.9, 71.1, 70.5, 61.3, 60.6, 51.7, 47.7, 35.8, 32.2, 30.3, 29.7, 29.3, 28.7, 28.1, 27.6, 13.0.

### Example 24: Synthesis of (Z)-((2S,5R)-5-((((trifluoromethyl)sulfonyl)oxy) methyl)-tetrahydrofuran-2-yl)methyl nonadec-9-enoate, B

Experimental: A flame dried, single neck, 25 mL round bottomed flask equipped with a PTFE coated magnetic stir bar was charged with 1.00 g of A (2.43 mmol), 590 L of pyridine (7.29 mmol), and 10 mL of anhydrous methylene chloride. The flask stoppered with a rubber septum and then immersed in a saturated brine/ice bath (˜−10° C.). While vigorously stirring, 409 uL of triflic anhydride (2.43 mmol) was added dropwise over a period of 15 min. After addition, the saline bath was removed and reaction continued overnight. The next morning a light yellow solution was observed; excess solvent and pyridine was removed under reduced pressure, and the dark yellow residue taken up in a minimum amount of chloroform, which was then charged to a prefabricated silica gel column that employed a hexanes/ethyl acetate gradient. The title compound B eluted at a 5:1 hexanes/ethyl acetate proportion, furnishing 663 mg of a loose yellow oil after inspissation (50% of theoretical). ¹H NMR (400 MHz, CDCl₃, salient cis isomer) δ (ppm) 5.59 (m, 1H), 5.52 (m, 1H), 4.38 (m, 2H), 4.17 (m, 1H), 4.07 (m, 1H), 3.92 (m, 1H), 3.79 (m, 1H), 2.27 (d, J=8.2 Hz, 2H), 2.26 (m, 1H), 2.24 (m, 1H), 1.90 (m, 2H), 1.67-1.65 (m, 4H), 1.36-1.30 (m, 22H), 1.01 (t, J=7.4 Hz, 3H); ¹³C NMR (100 MHz, CDCl₃, salient cis isomer) δ (ppm) 171.6, 128.5, 128.1, 114.6, 86.2, 85.7, 69.3, 62.7, 35.2, 33.5, 31.4, 32.6, 32.4, 32.2, 31.9, 31.8, 31.6, 30.5, 30.3, 30.2, 29.8, 294. 29.0, 28.7, 28.2, 21.0, 16.8.

### Example 25: Synthesis of ((2S,5R)-5-((((trifluoromethyl) sulfonyl)oxy) methyl)tetra-hydrofuran-2-yl)methyl tetradecanoate, B

Experimental: A flame dried, single neck, 25 mL round bottomed flask equipped with a PTFE coated magnetic stir bar was charged with 1.00 g of A (2.92 mmol), 709 L of pyridine (8.76 mmol), and 10 mL of anhydrous methylene chloride. The flask stoppered with a rubber septum and then immersed in a saturated brine/ice bath (˜−10° C.). While vigorously stirring, 491 uL of triflic anhydride (2.92 mmol) was added dropwise over a period of 15 min. After addition, the saline bath was removed and reaction continued overnight. The next morning a light yellow solution was observed; excess solvent and pyridine was removed under reduced pressure, and the dark yellow residue taken up in a minimum amount of chloroform, which was then charged to a prefabricated silica gel column that employed a hexanes/ethyl acetate gradient. The title compound B eluted at a 4:1 hexanes/ethyl acetate proportion, furnishing 607 mg of a loose colorless oil after inspissation (44% of theoretical). ¹H NMR (400 MHz, CDCl₃, salient cis isomer) δ (ppm) 4.36 (m, 2H), 4.27 (m, 2H), 4.15 (m, 1H), 4.05 (m, 1H), 3.93 (m, 1H), 3.84 (m, 1H), 2.21 (d, J=8.0 Hz, 2H), 1.94 (m, 2H), 1.64-1.62 (m, 2H), 1.28-1.24 (m, 20H), 0.94 (t, J=7.2 Hz, 3H); ¹³C NMR (100 MHz, CDCl₃, salient cis isomer) δ (ppm) 172.1, 115.2, 86.0, 85.6, 67.5, 63.3, 34.9, 31.9, 31.4, 30.6, 30.3, 30.0, 29.7, 29.3, 29.1, 28.8, 28.4. 28.0, 21.5, 15.3.

### Example 26: Synthesis of ((2S,5R)-5-(((2-(2-hydroxyethoxy)ethyl)amino) methyl)tetra-hydrofuran-2-yl)methyl oleate, B

Experimental: An oven dried, 50 mL round bottomed flask equipped with a PTFE coated magnetic stir bar was charged with 1.00 g of A (1.84 mmol), 212 mg of 2-(2-aminoethoxy)ethanol (2.02 mmol), and 25 mL of absolute ethanol. A reflux condenser was affixed to the neck, and, while vigorously stirring, the mixture was heated to 50° C. overnight. After this time, TLC (neutral alumina, isocratic ethanol eluent) indicated that all of A had been consumed, manifesting a single product with an R_{f}=0.52. The solution was then cooled to room temperature, then poured directly onto a prefabricated Brockmann I activated, neutral alumina column where B was observed to elute using an isocratic ethanol mobile phase. After drying under high vacuum for period of 7 days, 634 mg of B was retained as a viscous, yellow oil (71% of theoretical). Elemental analysis: Expected for C₂₈H₅₃NO₅—C, 69.52%; H, 11.04%. Found—C, 69.49%; H, 11.02%.

### Example 27: Synthesis of ((2S,5R)-5-(((2-(2-hydroxyethoxy)ethyl)amino) methyl)tetra-hydrofuran-2-yl)methyl tetradecanoate, B

Experimental: An oven dried, 50 mL round bottomed flask equipped with a PTFE coated magnetic stir bar was charged with 1.00 g of A (2.11 mmol), 244 mg of 2-(2-aminoethoxy)ethanol (2.32 mmol), and 25 mL of absolute ethanol. A reflux condenser was affixed to the neck, and, while vigorously stirring, the mixture was heated to 50° C. overnight. After this time, TLC (neutral alumina, isocratic ethanol eluent) indicated that all of A had been consumed, manifesting a single product with an R_{f}=0.49. The solution was then cooled to room temperature, then poured directly onto a prefabricated Brockmann I activated, neutral alumina column where B was observed to elute using an isocratic ethanol mobile phase. After drying under high vacuum for period of 7 days, 589 mg of B was retained as a viscous, colorless oil (65% of theoretical). Elemental analysis: Expected for C₂₄H₄₇NO₅—C, 67.09%; H, 11.03%. Found—C, 67.21%; H, 11.00%.

### Example 28: Synthesis of ((2S,5R)-5-(((2-((2-hydroxyethyl)amino)ethyl) amino)methyl)-tetrahydrofuran-2-yl)methyl tetradecanoate, B

Experimental: An oven dried, 50 mL round bottomed flask equipped with a PTFE coated magnetic stir bar was charged with 1.00 g of A (2.11 mmol), 242 mg of 2-((2-aminoethyl)amino)ethanol (2.32 mmol), and 25 mL of absolute ethanol. A reflux condenser was affixed to the neck, and, while vigorously stirring, the mixture was heated to 50° C. overnight. After this time, TLC (neutral alumina, isocratic ethanol eluent) indicated that all of A had been consumed, manifesting a single product with an R_{f}=0.33. The solution was then cooled to room temperature, then poured directly onto a prefabricated Brockmann I activated, neutral alumina column where B was observed to elute using an isocratic ethanol mobile phase. After drying under high vacuum for period of 7 days, 601 mg of B was retained as a viscous, intensely yellow oil (67% of theoretical). Elemental analysis: Expected for C₂₄H₄₈N₂O₄—C, 67.25%; H, 11.29%. Found—C, 67.09%; H, 11.20%.

### Example 29: ((2 S,5R)-5-(((2-((2-hydroxyethyl)amino)ethyl)amino)methyl) tetra-hydrofuran-2-yl)methyl oleate, B

Experimental: An oven dried, 50 mL round bottomed flask equipped with a PTFE coated magnetic stir bar was charged with 1.00 g of A (1.84 mmol), 210 mg of 2-((2-aminoethyl)amino)ethanol (2.02 mmol), and 25 mL of absolute ethanol. A reflux condenser was affixed to the neck, and, while vigorously stirring, the mixture was heated to 50° C. overnight. After this time, TLC (neutral alumina, isocratic ethanol eluent) indicated that all of A had been consumed, manifesting a single product with an R_{f}=0.38. The solution was then cooled to room temperature, then poured directly onto a prefabricated Brockmann I activated, neutral alumina column where B was observed to elute using an isocratic ethanol mobile phase. After drying under high vacuum for period of 7 days, 522 mg of B was retained as a viscous, yellow oil (59% of theoretical). Elemental analysis: Expected for C₂₈H₅₄N₂O₄—C, 69.66%; H, 11.27%. Found—C, 69.57%; H, 11.15%

### Example 30: Synthesis of (Z)-(5-((((trifluoromethyl)sulfonyl)oxy)methyl) furan-2-yl)methyl nonadec-9-enoate, B

Experimental: A flame dried, single neck, 25 mL round bottomed flask equipped with a PTFE coated magnetic stir bar was charged with 1.00 g of A (2.46 mmol), 597 L of pyridine (7.39 mmol), and 10 mL of anhydrous methylene chloride. The flask stoppered with a rubber septum and then immersed in a saturated brine/ice bath (˜−10° C.). While vigorously stirring, 419 μL of triflic anhydride (2.46 mmol) was added dropwise over a period of 15 min. After addition, the saline bath was removed and reaction continued overnight. The next morning a light yellow solution was observed; excess solvent and pyridine was removed under reduced pressure, and the dark yellow residue taken up in a minimum amount of chloroform, which was then charged to a prefabricated silica gel column that employed a hexanes/ethyl acetate gradient. The title compound B eluted at a 4:1 hexanes/ethyl acetate proportion, furnishing 701 mg of a loose, colorless oil after concentration (53% of theoretical). ¹H NMR (400 MHz, CDCl₃) δ (ppm) 6.25 (d, J=8.0 Hz, 1H), 6.22 (d, J=8.0 Hz, 1H), 5.55 (m, 1H), 5.52 (m, 1H), 5.18 (s, 2H), 4.55 (s, 2H), 2.44 (t, J=7.2 Hz, 2H), 2.07 (m, 2H), 1.57 (m, 2H), 1.35-1.29 (m, 22H), 1.00 (t, J=7.3 Hz, 3H). ¹³C NMR (100 MHz, CDCl₃) δ (ppm) 171.9, 153.3, 140.5, 129.4, 129.1, 115.1, 106.9, 106.4, 65.1, 61.9, 34.0, 32.4, 31.7, 31.5, 30.9, 30.5, 30.2, 30.0, 29.9, 29.7, 29.4, 29.3, 26.1, 22.9, 15.0.

### Example 31: Synthesis of (5-((((trifluoromethyl)sulfonyl)oxy)methyl)furan-2-yl)methyl dodecanoate, B

Experimental: A flame dried, single neck, 25 mL round bottomed flask equipped with a PTFE coated magnetic stir bar was charged with 1.00 g of A (3.22 mmol), 782 L of pyridine (9.66 mmol), and 10 mL of anhydrous methylene chloride. The flask stoppered with a rubber septum and then immersed in a saturated brine/ice bath (˜−10° C.). While vigorously stirring, 541 μL of triflic anhydride (3.22 mmol) was added dropwise over a period of 15 min. After addition, the saline bath was removed and reaction continued overnight. The next morning a light yellow solution was observed; excess solvent and pyridine was removed under reduced pressure, and the dark yellow residue taken up in a minimum amount of chloroform, which was then charged to a prefabricated silica gel column that employed a hexanes/ethyl acetate gradient. The title compound B eluted at a 4:1 hexanes/ethyl acetate proportion, furnishing 656 mg of a loose, colorless oil after concentration (46% of theoretical). ¹H NMR (400 MHz, CDCl₃) δ (ppm) 6.24 (d, J=8.0 Hz, 1H), 6.21 (d, J=8.0 Hz, 1H), 5.20 (s, 2H), 4.51 (s, 2H), 2.49 (t, J=7.2 Hz, 2H), 1.59 (dd, J=7.4 Hz, J=2.7 Hz, 2H), 1.30-1.27 (m, 16H), 1.09 (t, J=7.6 Hz, 3H). ¹³C NMR (100 MHz, CDCl₃) δ (ppm) 171.5, 153.0, 140.1, 115.4, 106.7, 106.3, 64.2, 60.9, 33.7, 30.8, 30.2, 30.1, 29.8, 29.7, 29.4, 29.3, 29.1, 29.0, 28.9, 24.7, 15.3.

### Example 32: Synthesis of (5-(((2-(2-hydroxyethoxy)ethyl)amino)methyl) furan-2-yl)methyl oleate, B

Experimental: An oven dried, 50 mL round bottomed flask equipped with a PTFE coated magnetic stir bar was charged with 1.00 g of A (1.91 mmol), 220 mg of 2-(2-aminoethoxy)ethanol (2.10 mmol), and 25 mL of absolute ethanol. A reflux condenser was affixed to the neck, and, while vigorously stirring, the mixture was heated to 50° C. overnight. After this time, TLC (neutral alumina, isocratic ethanol eluent) indicated that all of A had been consumed, manifesting a single product with an R_{f}=0.48. The solution was then cooled to room temperature, then poured directly onto a prefabricated Brockmann I activated, neutral alumina column where B was observed to elute using an isocratic ethanol mobile phase. After drying under high vacuum for period of 7 days, 682 mg of B was retained as a viscous, yellow oil (74% of theoretical). Elemental analysis: Expected for C₂₈H₄₉NO₅—C, 70.11%; H, 10.30%. Found—C, 70.03%; H, 10.22%.

### Example 33: Synthesis of (5-(((2-(2-hydroxyethoxy)ethyl)amino)methyl) furan-2-yl)methyl dodecanoate, B

Experimental: An oven dried, 50 mL round bottomed flask equipped with a PTFE coated magnetic stir bar was charged with 1.00 g of A (2.26 mmol), 261 mg of 2-(2-aminoethoxy)ethanol (2.49 mmol), and 25 mL of absolute ethanol. A reflux condenser was affixed to the neck, and, while vigorously stirring, the mixture was heated to 50° C. overnight. After this time, TLC (neutral alumina, isocratic ethanol eluent) indicated that all of A had been consumed, manifesting a single product with an R_{f}=0.46. The solution was then cooled to room temperature, then poured directly onto a prefabricated Brockmann I activated, neutral alumina column where B was observed to elute using an isocratic ethanol mobile phase. After drying under high vacuum for period of 7 days, 601 mg of B was retained as a viscous, colorless oil (67% of theoretical). Elemental analysis: Expected for C₂₂H₃₉NO₅—C, 66.47%; H, 9.89%. Found—C, 66.60%; H, 9.98%.

### Example 34: Synthesis of (5-(((2-((2-hydroxyethyl)amino)ethyl) amino)methyl) furan-2-yl)methyl oleate, B

Experimental: An oven dried, 50 mL round bottomed flask equipped with a PTFE coated magnetic stir bar was charged with 1.00 g of A (1.91 mmol), 218 mg of 2-((2-aminoethyl)amino)ethanol (2.10 mmol), and 25 mL of absolute ethanol. A reflux condenser was affixed to the neck, and, while vigorously stirring, the mixture was heated to 50° C. overnight. After this time, TLC (neutral alumina, isocratic ethanol eluent) indicated that all of A had been consumed, manifesting a single product with an R_{f}=0.32. The solution was then cooled to room temperature, then poured directly onto a prefabricated Brockmann I activated, neutral alumina column where B was observed to elute using an isocratic ethanol mobile phase. After drying under high vacuum for period of 7 days, 702 mg of B was retained as a viscous, deeply yellow oil (77% of theoretical). Elemental analysis: Expected for C₂₈H₅₀N₂O₄—C, 70.25%; H, 10.53%. Found—C, 70.16%; H, 10.59%.

### Example 35: Synthesis of (5-(((2-((2-hydroxyethyl)amino) ethyl)amino) methyl)furan-2-yl)methyl dodecanoate, B

Experimental: An oven dried, 50 mL round bottomed flask equipped with a PTFE coated magnetic stir bar was charged with 1.00 g of A (2.26 mmol), 259 mg of 2-((2-aminoethyl)amino)ethanol (2.49 mmol), and 25 mL of absolute ethanol. A reflux condenser was affixed to the neck, and, while vigorously stirring, the mixture was heated to 50° C. overnight. After this time, TLC (neutral alumina, isocratic ethanol eluent) indicated that all of A had been consumed, manifesting a single product with an R_{f}=0.28. The solution was then cooled to room temperature, then poured directly onto a prefabricated Brockmann I activated, neutral alumina column where B was observed to elute using an isocratic ethanol mobile phase. After drying under high vacuum for period of 7 days, 618 mg of B was retained as a viscous, pale yellow oil (69% of theoretical). Elemental analysis: Expected for C₂₂H₄₀N₂O₄—C, 66.63%; H, 10.17%. Found—C, 66.54%; H, 10.10%.

1-40. (canceled)

41) A furan or tetrahydrofuran compound selected from the group consisting of: 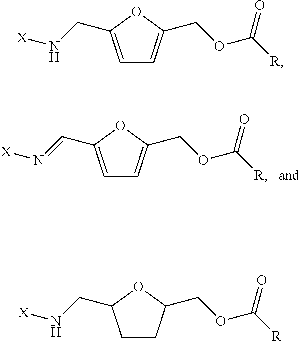
wherein R is a carbon side chain of a fatty acid with 8 to 30 carbons and X is an organic substituent having sufficient hydrogen bonding capacity to make the compound amphiphilic.

42) A furan or tetrahydrofuran compound selected from the group consisting of: 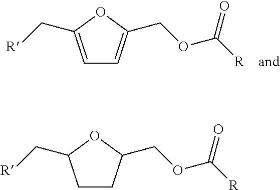
wherein R is a carbon side chain of a fatty acid with 8 to 30 carbons and R′ is a sulfonate ester moiety created by a sulfonating agent selected from the group consisting of: p-toluenesulfonyl (Tosyl), methanesulfonyl, (Mesyl), ethanesulfonate (Esyl), benzenesulfonate (Besyl), p-bromobenzenesulfonate (Brosyl), and triflouromethanesulfonic anhydride (triflate).

43) A method of making a furan imine compound of the formula 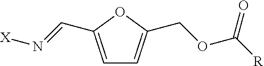
comprising contacting a HMF fatty acid ester with a primary amine to form the imine of the HMF fatty acid ester.

44) The method of claim 44, wherein said HMF fatty acid ester is made by contacting HMF with a fatty acid in the presence of a lipase enzyme.

45) The method of claim 45, wherein said lipase enzyme is Candida Antarctica B lipase.

46) The method of claim 44, wherein the contacting of the HMF fatty acid ester is carried out at a temperature of about 30° C. to about 100° C.

47) The method of claim 45, wherein the contacting of the HMF fatty acid ester done in the presence of a polar solvent selected from the group consisting of: acetonitrile, dimethylformamide, dimethylacetamide, dimethylsulfoxide, isopropanol, methanol, and ethanol.

48) The method of claim 45, wherein further the imine compound is reduced with a reducing agent to form a corresponding amine of the HMF fatty acid ester.

49) The method of claim 50, wherein said reducing agent is a hydride.

50) The method of claim 51, wherein said hydride is a borohydride selected from the group consisting of: sodium cyanoborohydride, lithium borohydride, calcium borohydride, magnesium borohydride, and sodium borohydride.

51) The method of claim 50, wherein the reduction of said imine compound is done at a temperature from about −20° C. to about 26° C.

52) The method of claim 53, wherein the reduction of said imine compound is done at a temperature from about −10° C. to about 10° C.

53) The method of claim 50, wherein said reduction is done in the presence of a polar solvent selected from the group consisting of: dimethylsulfoxide, dimethylformamide, dimethylacetamide, methanol, ethanol, isopropanol, tetrahydrofuran, and acetone.

54) A method of making a compound of the formula selected from the group consisting of: 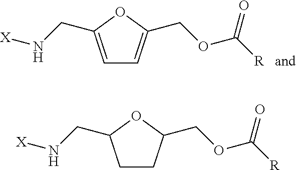
comprising;
a) contacting a dihydroxymethylfuran or a tetrohydrofuran fatty acid ester with a sulfonating agent to form a sulfonate ester moiety making a sulfonated fatty acid ester; and
b) contacting the sulfonated ester moiety of the sulfonated fatty acid ester with a primary amine to displace said sulfonated ester moiety with the primary amine in the presence of a nucleophilic base selected from the group consisting of dimethylaminopyridine, imidazole, pyrazole, and pyridine.

55) The method of claim 56, wherein said dihydroxymethylfuran or tetrohydrofuran fatty acid ester is made by contacting the dihydroxymethylfuran or tetrahydrofuran with a fatty acid in the presence of a lipase enzyme.

56) The method of claim 57, wherein said lipase enzyme is Candida Antarctica B lipase.

57) The method of claim 56, wherein the contacting of said dihydroxymethylfuran or tetrohydrofuran fatty acid ester with the sulfonating agent is done in the presence of an organic solvent selected from the group consisting of: chloroform, tetrahydrofuran, acetone, benzene, diethyl ether, and methylene chloride.

58) The method of claim 56, wherein said dihydroxymethylfuran or tetrohydrofuran fatty acid ester is contacted with the sulfonating agent to form a sulfonate ester at a temperature of from about −20° C. to about 26° C.

59) The method of claim 56, wherein the contacting of said sulfonated fatty acid ester with the primary amine is done in the presence of a polar solvent selected from the group consisting of: dimethylsulfoxide, dimethylformamide, dimethylacetamide, N-methylpyrrolidone, acetonitrile, methanol, ethanol, and acetone.

60) The method of claim 56, wherein said sulfonated fatty acid ester is contacted with said primary amine at a temperature from about 30° C. to about 100° C.
